(19) United States
(12) Patent Application Publication
(10) Pub. No.: US 2003/0092028 A1
(43) Pub. Date: May 15, 2003

(54) Methods and Reagents For Diagnosis and Treatment of Insulin Resistance and Related Condition

(76) Inventors: Yuanhong MA, Los Altos, CA (US); Chih-Jian LIH, Mountain View, CA (US); Fan CHEN, USA, CA (US); Jeffery FAIRMAN, Mountain View, CA (US); Yii-Der CHEN, Saratoga, CA (US)

Correspondence Address: Morrison, Foerster, 755 Page Mill Road, Palo Alto, CA, 94304, rtapple@mofo.com

(21) Appl. No.: 10/161,803

(22) Filed: Jun. 3, 2002

Related U.S. Application Data
(63) Continuation of application No. 60/295,264.
(60) Provisional application No. 60/295,264, filed on Jun. 3, 2002.

(51) Int. Cl. C12Q 1/68

(52) U.S. Cl. 435/6

(57) ABSTRACT

### Abstract of Disclosure

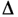

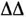

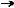

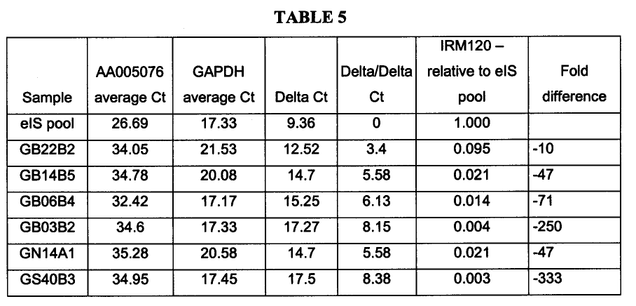

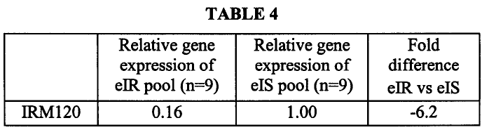

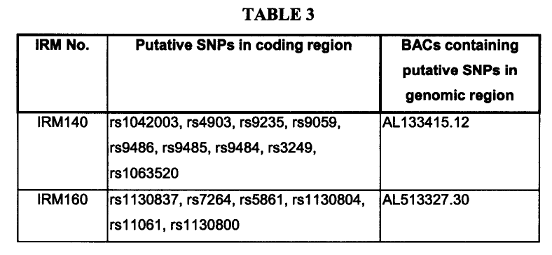

METHODS AND REAGENTS FOR DIAGNOSIS AND TREATMENT OF INSULIN RESISTANCE AND RELATED CONDITIONSMethods, reagents and devices for diagnosis, prognosis and treatment of insulin resistance and insulin resistance related conditions are provided. Methods for identification of agents useful in treatment of insulin resistance and insulin resistance related conditions, and agents so identified, are provided

### Cross Reference to Related Applications

This application claims benefit of provisional patent application no. 60/295,264, filed June 1, 2001. The entire contents of the provisional application are incorporated herein by reference for all purposes.

### Field of the Invention

The present invention related to diagnosis and treatment of insulin resistance and related conditions. The invention finds use in the fields of medicine and biology.

### Background of the Invention

The maintenance of glucose homeostasis in humans involves dynamic balances among glucose absorption in the gut, glucose utilization by brain, muscle and adipose tissue, glucose sensing and insulin secretion by pancreas, and glucose synthesis and storage by liver (for a review, see Shepherd and Kahn, 1999, New England J Med. 341:248-57). Blood glucose levels are regulated by complex interactions involving circulating hormones (primarily insulin and glucagon), cellular proteins involved in insulin signaling and glucose transport, and multiple genetic factors yet to be identified. Resistance to insulin-stimulated glucose uptake in insulin-responsive tissues (muscle and adipose tissue) is considered the primary cause of type II diabetes that affects more than 150 millions individuals worldwide. In addition, insulin resistance (IR) represents a common biochemical abnormality that occurs in up to 25% of the general population, and has been strongly associated with a cluster of metabolic diseases, termed Syndrome X (insulin resistance syndrome) that include reduced levels of circulating high-density lipoproteins, hypertension, abdominal obesity and coronary artery disease (Reaven, Diabetes 37:1595-1607 (1988); De Fronzo et al., Diabetes Care 14:173-194 (1991); Reaven, Metabolism 41:16-19 (1992)). All of these are known to be the major contributors of mortality and morbidity in developed countries (Reaven, 1994, J Internal. Medicine 236:13-22)

It is clear, based on genetic evidence, that insulin resistance is due to genetic defects in a variety of genes in functionally-related pathways, although many key genes in these pathways remain poorly understood (Pedersen, 1999, Exp Clin Endocrinal Diabetes 107:113-118). Intense research over the past two decades has led to the discovery of genes for insulin, insulin receptor, insulin receptor substrates, phosphatidylinositol-3(PI3)-kinase, glucose transporters, glycogen synthase, and glucokinase. However, mutations in these genes are rare, accounting only for a small portion (<1%) of IR-related syndromes. Thus, there is a need to identify additional genes and proteins associated with insulin resistance and related conditions.

### Summary of Invention

The invention relates to insulin resistance markers (IRMs). IRM genes are differentially expressed in insulin resistant individuals compared to normal or insulin sensitive individuals. Insulin resistance markers of the invention are listed in Table 1. IRM genes encode RNAs (IRM gene products) that hybridize (e.g., under stringent conditions) to a polynucleotide having the sequence of, or exactly complementary to, a sequence identified in Table 1 by GenBank accession number.

In one aspect, the invention provides a method of determining whether a subject is at risk of developing insulin resistance by detecting a difference in sequence of an IRM gene, or a difference in expression of an IRM gene product, in a biological sample from an insulin resistant subject and a biological sample from a non-insulin resistant subject. In various embodiments the non-insulin resistant subject has an eIS phenotype and/or the insulin resistant subject has an eIR phenotype. In one embodiment, the method involves detecting a difference in sequence of at least 2, optionally at least 3, 4, 5, 6, 7, 8, 9, 10, 11, 12, 15, 20, or at least 25 IRM genes or detecting a difference in expression of at least 2, optionally at least 3, 4, 5, 6, 7, 8, 9, 10, 11, 12, 15, 20, or at least 25 IRM gene products.

In one embodiment, the invention provides a method for diagnosing for insulin resistance (IR), IR-related conditions, or susceptibility to IR or IR-related conditions in a subject, by detecting a difference in expression of at least one insulin resistance marker (IRM) listed in Table 1 in a biological sample from the subject, compared to the level of expression of the IRM characteristic of expression in a similar biological sample in a reference population of individuals who are not insulin resistant.

In one embodiment, the invention provides a method for diagnosing for insulin resistance (IR), IR-related conditions, or susceptibility to IR or IR-related conditions in a subject by determining the level of expression of at least one insulin resistance marker (IRM) listed in Table 1 in a biological sample from the subject, and detecting a difference (e.g., an increase or decrease) in expression compared to the level of expression of the IRM characteristic of expression in a similar biological sample in a reference population of individuals who are not insulin resistant (e.g., individuals with an eIS phenotype).

In a related aspect, the invention provides a method of determining whether a subject is insulin resistant or at risk of developing insulin resistance by providing a biological sample of the subject and comparing the level of expression of an IRM gene product in the sample to the level of expression characteristic of a sample of the same type in a healthy individual or population, where a difference in the sample from the subject is an indication that the individual is insulin resistant or at risk of developing insulin resistance. In another related aspect, the invention provides a method of determining whether an individual is insulin resistant by identifying a patient at risk for insulin resistance, providing a biological sample of the subject and comparing the level of expression of an IRM gene product in the sample to the level of expression characteristic of a sample of the same type in a healthy individual or population, where a difference in the sample from the subject is an indication that the individual is insulin resistant. In various embodiments, the IRM gene product is detected by amplification (for example, using a primer with at least 10 contiguous bases, optionally at least 15 contiguous bases, identical to or exactly complementary to an accession sequence), by hybridization (for example, using a probe with at least 10 contiguous bases, optionally at least 15 contiguous bases, identical to, or exactly complementary to, an accession sequence), or by detecting an IRM polypeptide. The biological sample may be a tissue sample, and is preferably from blood, e.g., a blood fraction such as blood cells (e.g., leukocytes, e.g. B cells).

In some embodiments, a panel of IRM genes is assayed for changes in expression or for the presence of polymorphisms. In one embodiment, at least 2 different IRMs are assayed for each subject. In other embodiments, at least 4, 5, 6, 7, 8, 9, 10, 11, 12, 15, 20, or 25 IRM genes or gene products are assayed. In one embodiment the invention provides a method of determining whether an individual is insulin resistant or at risk for developing insulin resistance by obtaining a biological sample taken from the subject, comparing the expression level of a panel of least 3, optionally at least 4, 5, 6, 7, 8, 9, 10, 11, 12, 15, 20, or 25 IRM genes in the sample to a reference value representative of expression in an individual (e.g., population of individuals) of a known insulin resistance status, and determining that the individual is insulin resistant or at risk for developing insulin resistance when the expression level of at least 25%, 50%, or 75% of the IRM genes is statistically similar to reference value, if the reference value is characteristic of expression in a subject who is insulin resistant or at risk for developing insulin resistance, or determining that the individual is insulin resistant or at risk for developing insulin resistance when the expression level of at least 25%, 50%, or 75% of the IRM genes is different from a the reference value, if the reference value is characteristic of expression in a healthy subject.

In one aspect, the invention provides a method for identifying a polymorphism associated with an insulin resistance (IR) phenotype or risk of developing insulin resistance by comparing the sequence of an IRM gene in a biological sample from an insulin resistant subject with sequence of an IRM gene in a biological sample from a non-insulin resistant subject. In various embodiments, the non-insulin resistant subject has an eIS phenotype and/or the insulin resistant subject has an eIR phenotype. In an embodiment, a mutation is identified in an intron, an exon, or a promoter region of the IRM gene. In an embodiment, a single base mutation the IRM gene is identified.

In one aspect, the invention provides a method of screening for an agent to determine its usefulness in treating insulin resistance by providing a cell expressing an IRM gene product, contacting the cell with a test agent, and determining whether the level of expression of the IRM gene product is changed in the presence of the test agent, wherein a change is an indication that the test agent is useful in treatment of insulin resistance. In various embodiments, the step of contacting the cell involves administering the test agent to an animal (e.g., an experimental model for diabetes, insulin resistance, insulin sensitivity, or an insulin resistance related condition. In various embodiments, the screening method involves determining whether the level of more than one IRM gene is affected by the agent. In one aspect, the invention provides a method of screening an agent or collection of test agents to determine its usefulness in treating insulin resistance by providing a composition comprising an IRM protein, contacting the composition with a test agent, and determining whether the activity of the IRM protein is changed in the presence of the test product, where a change is an indication that the test agent is useful in treating insulin resistance.

In another aspect, the invention provides a method of treating insulin resistance in a mammal by administering an effective amount of an agent that modulates expression of an IRM gene product (e.g., where the IRM gene product is an RNA that hybridizes under stringent conditions to a polynucleotide having the sequence of, or exactly complementary to, an accession sequence). In an embodiment, the invention provides a method of treating insulin resistance in a mammal, comprising administering an effective amount of an agent that modulates expression of an IRM gene product. In various embodiments, the agent results in an increase in expression or activity of the IRM gene product or results in a decrease in expression or activity of the IRM gene product. In an embodiment, the mammal is a human subject suffering from symptoms or complications of insulin resistance or a condition related to insulin resistance. In a related aspect, the invention provides the use of an agent that modulates expression of an IRM gene product in the formulation of a pharmaceutical composition for the treatment of IR.

In another aspect, the invention provides kits for diagnosis of insulin resistance (and related conditions) or screening for agents useful for treatment of insulin resistance (and related conditions). In one embodiment, the kit includes probes (e.g., polynucleotide or antibody probes) specific for a plurality of different IRM gene products. In a related embodiment, the kit includes a substrate on which a plurality of IRM probes or gene products are immobilized.

In another aspect, the invention provides a method for identifying a polymorphism associated with an insulin resistance (IR) phenotype or risk of developing insulin resistance by comparing the sequence of an IRM gene listed in Table 1 in a biological sample from an insulin resistant subject with sequence of the IRM gene in a biological sample from a non-insulin resistant subject. In an embodiment, the non-insulin resistant subject has an eIS phenotype. In an embodiment, the insulin resistant subject has an eIR phenotype.

In a related aspect, the invention provides a method of determining whether an individual is at risk of developing insulin resistance or whether said individual suffers from insulin resistance by (a) obtaining a nucleic acid sample from said individual; and (b) determining whether the nucleotides present at one or more IRM genes are indicative of a risk of developing insulin resistance. Further provided is a method of detecting an association between a genotype and an insulin resistance phenotype, by (a) genotyping at least one IRM gene in a first population having a first insulin resistance phenotype; (b) genotyping said IRM gene in a second population having a second insulin resistance phenotype different from the first insluin resistance phenotype; and (c) determining whether a statistically significant association exists between said genotype and said phenotype. In an embodiment, the first population is eIS and second population is eIR.

In a related aspect, the invention provides a method of estimating the frequency of a haplotype for a set of nucleotide polymorphisms markers in a population, by (a) identifying at least a first nucleotide polymorphism in an IRM gene listed in Table 1 for individuals in a population; (b) identifying a second nucleotide polymorphism in an IRM gene for individuals in a population, wherein the second IRM gene is the same or different from the first IRM gene; and (c) applying an haplotype determination method to the identities of the nucleotide polymorphisms determined in steps (a) and (b) to obtain an estimate of said frequency.

In a different aspect, the invention provides a method for identifying a gene expression pattern diagnostic of a disease state by identifying a first population of human subjects, where the subjects suffer from, or are at high risk of, developing the disease, identifying a second population of human subjects, where the subjects are at low risk of developing the disease; and identifying at least 3 RNA sequences differentially expressed in the first population compared to the second population. In one embodiment, the invention comprises obtaining cell lines derived from B lymphocytes from each of the subjects in the first and second populations and identifying genes that are differentially expressed in one cell line compared to another. In one embodiment, the cell lines are derived from blood cells. For example, the cell lines may be derived from Epstein Barr virus transformed B cells. Generally, the first and second populations each comprise at least 3 individuals, and often at least 5 individuals or more. In one embodiment, the step of identifying differentially expressed RNA sequences includes i) obtaining cell lines derived from a tissue from each of the subjects in the first and second populations; ii) obtaining RNA from said cell lines, iii) preparing a pooled probe corresponding the RNA from each cell line; and iv) hybridizing the pooled probe to a nucleic acid array comprising a plurality expressed sequence tags (cDNAs) from the tissue. In one embodiment, the nucleic acid array has at least 100 different expressed sequence tags.

### Detailed Description

GENERAL REFERENCES & DEFINITIONS References The following references provide information useful in the practice of the invention: (1) Sambrook et al. (1989) Molecular Cloning: A Laboratory Manual (2nd Edition) Cold Spring Harbor Laboratory Press and Sambrook and Russel (2001) Molecular Cloning: A Laboratory Manual (3rd Edition) Cold Spring Harbor Laboratory Press (hereinafter, referred to together or individually as "Sambrook"); (2) Ausubel et al. (1987) Current Protocols In Molecular Biology (as supplemented through 2001), John Wiley & Sons, New York (hereinafter, "Ausubel"); (3) Coligan et al., Current Protocols In Immunology (as supplemented through 2001), John Wiley & Sons, New York (hereinafter, "Coligan"); (4) Harlow and Lane (1988) Antibodies, A Laboratory Manual, Cold Spring Harbor Publications, New York, and Harlow and Lane (1999) Using Antibodies: A Laboratory Manual Cold Spring Harbor Laboratory Press, Cold Spring Harbor, NY (hereinafter, referred to together or individually as "Harlow and Lane"). (5) Current Protocols in Immunology (J.E. Coligan et al., eds., 1999, including supplements through 2001); (6) PCR: The Polymerase Chain Reaction, (Mullis et al., eds., 1994); (7) Bioconjugate Techniques (Greg T. Hermanson, ed., Academic Press, 1996); and (8) Beaucage et al. eds., Current Protocols in Nucleic Acid Chemistry John Wiley & Sons, Inc., New York, 2000).

Definitions The following definitions are provided to assist the reader in the practice of the invention:

The terms "allele"or "allelic sequence,"as used herein, refer to a naturally-occurring alternative form of a gene encoding a specified polypeptide (i.e., an IRM gene sequence).

The term "antibody,"as used herein refers to specific binding molecules comprising V_{L} and/or V_{H} sequences, including, for example (i) polyclonal antibody preparations, (ii) monoclonal antibodies (iii) (vi) humanized antibody molecules (see, for example, Riechmann et al. (1988) Nature 332:323-327; Verhoeyan et al. (1988) Science 239:1534-1536; (iv) hybrid (chimeric) antibody molecules (see, for example, Winter et al. (1991) Nature 349:293-299; and U.S. Patent No. 4,816,567); (v) antibody fragments, e.g., F(ab")2 and F(ab) fragments; (vi) Fv molecules (noncovalent heterodimers, see, for example, Ehrlich et al. (1980) Biochem 19:4091-4096); (vi) single-chain Fv molecules (sFv) (see, for example, Huston et al. (1988) Proc. Natl. Acad. Sci. USA 85:5879-5883); (vii)and trimeric antibody fragment constructs; (viii) Mini-antibodies or minibodies (i.e., sFv polypeptide chains that include oligomerization domains at their C-termini, separated from the sFv by a hinge region; see, e.g., Pack et al. (1992) Biochem 31:1579-1584; and, (ix) any functional fragments obtained from such molecules, wherein such fragments retain specific-binding properties of the parent antibody molecule.

The term "antisense sequences"refers to polynucleotides having sequence complementary to a RNA sequence. These terms specifically encompass nucleic acid sequences that bind to mRNA or portions thereof to block transcription of mRNA by ribosomes. Antisense methods are generally well known in the art (see, e.g., PCT publication WO 94/12633, and Nielsen et al., 1991, Science 254:1497; Oligonucleotides and Analogues, A Practical Approach, edited by F. Eckstein, IRL Press at Oxford University Press (1991); Antisense Research and Applications (1993, CRC Press)).

The term "conservative substitution,"when describing a polypeptide, refers to a change in the amino acid composition of the polypeptide that does not substantially alter the activity of the polypeptide, i.e., substitution of amino acids with other amino acids having similar properties (e.g., acidic, basic, positively or negatively charged, polar or non-polar, etc.) such that the substitutions of even critical amino acids does not substantially alter activity. Conservative substitution tables providing functionally similar amino acids are well known in the art. The following six groups each contain amino acids that are conservative substitutions for one another: 1)(A), Serine (S), Threonine (T); 2)acid (D), Glutamic acid (E); 3)(N), Glutamine (Q); 4)(R), Lysine (K); 5)(I), Leucine (L), Methionine (M), Valine (V); and 6)(F), Tyrosine (Y), Tryptophan (W) (see also, Creighton, 1984, Proteins, W.H. Freeman and Company).

The term "detectably labeled"means that an agent (e.g., a probe) has been conjugated with a label that can be detected by spectroscopic, photochemical, biochemical, immunochemical, electrical, optical, electromagnetic and other related analytical techniques. Examples of detectable labels that can be utilized include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates. The term "labeled", with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term "epitope"has its ordinary meaning of a site on an antigen recognized by an antibody. Epitopes are typically segments of amino acids which are a small portion of the whole polypeptide. Epitopes may be conformational (i.e., discontinuous). That is, they may be formed from amino acids encoded by noncontiguous parts of a primary sequence that have been juxtaposed by protein folding.

The term "gene product"refers to an RNA molecule transcribed from a gene, or a polypeptide encoded by the gene or translated from the RNA.

The term "kit" refers to components packaged or marked for use together. For example, a kit can contain multiple polynucleotide or antibody probes in separate containers. Alternatively, a kit can contain any two components in one container, and a third component and any additional components in one or more separate containers. Optionally, a kit further contains instructions for combining the components.

The term "naturally occurring"as applied to a compound or composition (e.g., an mRNA) means that the compound or composition can be found in nature.

The terms "nucleic acid,""polynucleotide,"and "oligonucleotide"are used herein to include a polymeric form of nucleotides of any length, including, but not limited to, ribonucleotides or deoxyribonucleotides. There is no intended distinction in length between these terms. Further, these terms refer only to the primary structure of the molecule. Thus, in certain embodiments these terms can include triple-, double- and single-stranded DNA, as well as triple-, double- and single-stranded RNA. They also include modifications, such as by methylation and/or by capping, and unmodified forms of the polynucleotide. More particularly, the terms "nucleic acid,""polynucleotide,"and "oligonucleotide," include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oregon, as Neugene) polymers, and other synthetic sequence-specific nucleic acid polymers providing that the polymers contain nucleobases in a configuration which allows for base pairing, such as is found in DNA and RNA. Unless otherwise specified, reference to a polynucleotide sequence is also intended to refer to the exact complement of the sequence, as determined by standard base-pairing rules, i.e., A 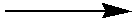 (T/U) and G 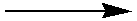 C. Thus, unless otherwise indicated, a statement that a reference polynucleotide sequence hybridizes to a second polynucleotide sequence is understood to encompass specific hybridization between either strand of the reference sequence to either strand of the second sequence.

The term "operably linked"refers to functional linkage between a nucleic acid expression control sequence (such as a promoter, signal sequence, or array of transcription factor binding sites) and a second polynucleotide, wherein the expression control sequence affects transcription and/or translation of the second polynucleotide. Generally, sequences that are operably linked are contiguous, and in the case of a signal sequence both contiguous and in reading phase. However, enhancers need not be located in close proximity to the coding sequences whose transcription they enhance.

By "pharmaceutically acceptable"it is meant the carrier, diluent or excipient must be compatible with the other ingredients of the formulation and not deleterious to the recipient thereof.

The term "polypeptide"is used interchangeably herein with the term "protein,"and refers to a polymer composed of amino acid residues linked by amide linkages, including synthetic, naturally-occurring and non-naturally occurring analogs thereof (amino acids and linkages). Peptides are examples of polypeptides.

A "primer"is a single-stranded polynucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. A primer need not reflect the exact sequence of the template but must be sufficiently complementary to hybridize with a template. The term "primer pair"means a set of primers including a 5" "upstream primer"that hybridizes with the complement of the 5" end of the DNA sequence to be amplified and a 3" "downstream primer"that hybridizes with the 3" end of the sequence to be amplified. A primer that is "perfectly complementary" has a sequence fully complementary across the entire length of the primer and has no mismatches. A "mismatch" refers to a site at which the nucleotide in the primer and the nucleotide in the target nucleic acid with which it is aligned are not complementary. The term "substantially complementary" when used in reference to a primer means that a primer is not perfectly complementary to its target sequence; instead, the primer is only sufficiently complementary to hybridize selectively to its respective strand at the desired primer-binding site. Primers are generally approximately 7 nucleotides or greater, and as many as approximately 100 nucleotides, often between about 10 and about 50 nucleotides in length, more often between about 12 and about 50 nucleotides, and very often between about 15 and about 25 nucleotides.

As used herein, a "probe,"when used in the context of polynucleotides and antibodies, refers to a molecule that specifically binds another molecule. One example of a probe is a "nucleic acid probe," which can be a DNA, RNA, or other polynucleotide. Where a specific sequence for a nucleic acid probe is given, it is understood that the complementary strand is also identified and included. The complementary strand will work equally well in situations where the target is a double-stranded nucleic acid that specifically binds (e.g., anneals or hybridizes) to a substantially complementary nucleic acid. Another example of a probe is an "antibody probe"that specifically binds to a corresponding antigen or epitope. A "cDNA probe" is prepared by reverse transcription of RNA (e.g. a single species or a heterogeneous population).

The term "recombinant"refers to a polynucleotide synthesized or otherwise manipulated in vitro (e.g., "recombinant polynucleotide"), to methods of using recombinant polynucleotides to produce gene products in cells or other biological systems, or to a polypeptide ("recombinant protein") encoded by a recombinant polynucleotide. Thus, a "recombinant" polynucleotide is defined either by its method of production or its structure. In reference to its method of production, the process is use of recombinant nucleic acid techniques, e.g., involving human intervention in the nucleotide sequence, typically selection or production. Alternatively, it can be a polynucleotide made by generating a sequence comprising fusion of two fragments which are not naturally contiguous to each other, but is meant to exclude products of nature. Thus, for example, products made by transforming cells with any non-naturally occurring vector is encompassed, as are polynucleotides comprising sequence derived using any synthetic oligonucleotide process. Similarly, a. "recombinant"polypeptide is one expressed from a recombinant polynucleotide. The term "recombinant" when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

The phrase "selectively hybridizing to"refers to a polynucleotide probe that hybridizes, duplexes or binds to a particular target DNA or RNA sequence when the target sequences are present in a preparation of total cellular DNA or RNA.

The phrases "specifically binds"when referring to a protein, "specifically immunologically cross reactive with,"or simply "specifically immunoreactive with"when referring to an antibody, refers to a binding reaction which is determinative of the presence of the protein in the presence of a heterogeneous population of proteins and other biologics. Thus, under designated conditions, a specified ligand binds preferentially to a particular protein and does not bind in a significant amount to other proteins present in the sample. A molecule or ligand (e.g., an antibody) that specifically binds to a protein has an association constant of at least 10³ M⁻¹ or 10⁴ M⁻¹, sometimes 10⁵ M⁻¹ or 10⁶ M⁻¹, in other instances 10⁶ M⁻¹ or 10⁷ M⁻¹, preferably 10⁸ M⁻¹ to 10⁹ M⁻¹, and more preferably, about 10¹⁰ M to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane for a description of immunoassay formats and conditions that can be used to determine specific immunoreactivity.

As used herein, the "substantial sequence identity,"refers to two or more sequences or subsequences that have at least 60%, preferably 80%, most preferably 90%, 95%, 98%, or 99% nucleotide or amino acid residue identity, when compared and aligned for maximum correspondence, as measured using one of the following sequence comparison algorithms or by visual inspection. Two sequences (amino acid or nucleotide) can be compared over their full-length (e.g., the length of the shorter of the two, if they are of substantially different lengths) or over a subsequence such as at least about 50, about 100, about 200, about 500 or about 1000 contiguous nucleotides or at least about 10, about 20, about 30, about 50 or about 100 contiguous amino acid residues. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally Ausubel et al.). Each of these references and algorithms is incorporated by reference herein in its entirety. When using any of the aforementioned algorithms, the default parameters for "Window" length, gap penalty, etc., are used. One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=-4, and a comparison of both strands.

Another indication that two nucleic acid sequences are substantially identical is that the two molecules hybridize to each other under stringent conditions. Substantial identity exists when the segments will hybridize under stringent hybridization conditions to a strand, or its complement, typically using a sequence of at least about 50 contiguous nucleotides derived from the probe nucleotide sequences. "Bind(s)" refers to complementary hybridization between a probe nucleic acid and a target nucleic acid and embraces minor mismatches that can be accommodated by reducing the stringency of the hybridization media to achieve the desired detection of the target polynucleotide sequence.

"Stringent hybridization conditions"refers to conditions in a range from about 5ºC to about 20ºC or 25ºC below the melting temperature (Tm) of the target sequence and a probe with exact or nearly exact complementarity to the target. As used herein, the melting temperature is the temperature at which a population of double-stranded nucleic acid molecules becomes half-dissociated into single strands. Methods for calculating the Tm of nucleic acids are well known in the art (see, e.g., Berger and Kimmel, 1987, Methods In Enzymology, Vol. 152: Guide To Molecular Cloning Techniques, San Diego: Academic Press, Inc. and Sambrook; supra. As indicated by standard references, a simple estimate of the Tm value may be calculated by the equation: Tm = 81.5 + 0.41 (% G + C), when a nucleic acid is in aqueous solution at 1 M NaCl (see e.g., Anderson and Young, "Quantitative Filter Hybridization"in Nucleic Acid Hybridization (1985)). Other references include more sophisticated computations which take structural as well as sequence characteristics into account for the calculation of Tm. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook, supra, and Ausubel, supra. Typically, stringent hybridization conditions are salt concentrations less than about 1.0 M sodium ion, typically about 0.01 to 1.0 M sodium ion at pH 7.0 to 8.3, and temperatures at least about 30ºC for short probes (e.g., 10 to 50 nucleotides) and at least about 60ºC for long probes (e.g., greater than 50 nucleotides). As noted, stringent conditions may also be achieved with the addition of destabilizing agents such as formamide, in which case lower temperatures may be employed.

The terms "substantially pure"or "isolated," when referring to proteins and polypeptides, denote those polypeptides that are separated from proteins or other contaminants with which they are naturally associated. A protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition. When referring to polynucleotides, the terms "substantially pure"or "isolated"generally refer to the polynucleotide separated from contaminants with which it is generally associated, e.g., lipids, proteins and other polynucleotides. The substantially pure or isolated polynucleotides of the present invention will be greater than about 50% pure. Typically, these polynucleotides will be more than about 60% pure, more typically, from about 75% to about 90% pure and preferably from about 95% to about 98% pure.

The term "therapeutically effective amount" means the amount of the subject compound that will elicit the biological or medical response of a tissue, system, animal or human that is being sought by the researcher, veterinarian, medical doctor or other clinician, e.g., ameliorate a disease state or symptoms, or otherwise prevent, hinder, retard or reverse the progression of a disease or any other undesirable symptoms. Amelioration of insulin resistance in a subject can be assayed by improved OGTT and SSPG profiles. A "prophylactic amount" is an amount sufficient to prevent, hinder or retard development or progression of the disease.

II.INTRODUCTIONThe present invention is based, in part, on the discovery of a convincing correlation between insulin resistance in humans and the expression pattern in blood cells of certain genes referred to herein as "insulin resistance marker genes"or "IRM genes." This correlation between IRM gene expression and insulin resistance was identified by large scale-gene expression profiling of two populations, an extreme insulin resistance population and an extreme insulin sensitivity population.

Approximately 600 subjects with at least one parent with type II diabetes were previously identified and screened for extreme insulin resistance ("eIR") or extreme insulin sensitivity ("eIS") using an oral glucose tolerance test and a steady-state plasma glucose test. An Oral Glucose Tolerance Test (OGTT) is an assessment of insulin sensitivity in vivo. See, e.g., Bergman et al., Endocrinology Review 6:45-86 (1985). In general, individuals with 75-g 2-hr OGTT > 140 mg/dl are considered insulin resistant, and individuals with 75-g 2-hr OGTT < 120 mg/dl are considered normal. A Steady State Plasma Glucose Test (SSPG) is a modification of the insulin-suppression test, in which subjects receive a continuous intravenous infusion of somatostatin, insulin and glucose. Reaven et al., Diabetologia 16:17-24 (1979). In general, individuals with SSPG mean >180 mg/dl are considered as insulin resistant; individuals with SSPG mean < 150 mg/dl are considered normal.

Subjects were assigned to the eIR group (i.e., an eIR phenotype) if they were over 18 years of age and met the following criteria: OGTT Glucose at 120 min (OGTT glucose level at 120 min after 75 g oral glucose load) > 140 mg/dl; SSPG mean > 250 mg/dl; OGTT Ins at 60 m > 100 µ IU/ml Et; OGTT Ins at 120 m > 100 µIU/ml. Subjects were assigned to the eIS group (i.e., an eIS phenotype) if they were over 18 years of age and met the following criteria: OGTT Glu at 120m < 100 mg/dl; SSPG mean < 120 mg/dl; OGTT Ins at 60m < 60 µ IU/m1 OR OGTT Ins at 120 m < 40 µ IU/ml. "SSPG mean" refers to the steadystate plasma glucose level (the average of the four values obtained at 150, 160, 170 and 180 min during a SSPG test). As used herein, "OGTT Ins at Xm"means the OGTT insulin level at X minutes after a 75 gram oral glucose load.

Established Epstein Barr Virus ("EBV")-transformed B lymphocyte cell lines from 6 eIR and 6 eIS age and gender matched subjects (i.e., 12 cell lines) were obtained. Assays using 50 known insulin-responsive genes had demonstrated that EBVB lymphocyte cell lines exposed to insulin exhibited a gene expression pattern similar to expression in pancreas, a classical "insulin-action"tissue.

The 12 cell lines were grown in the presence of 15 µ IU/ml or 100 µ IU/ml of insulin under the same culture conditions to the same passages, and total RNA was extracted from each cell line (IU = international unit). Equal amounts of the total RNA from the 6 eIR cell lines were pooled to form the eIR-RNA pool and equal amounts of the total RNA from 6 eIS cell lines were pooled to form the eISpool. Differently labeled probes were prepared by reverse transcription with oligo-dT primer to specifically amplify mRNA of the eIR and eIS pools. The probes were hybridized to microarrays containing approximately 10,000 expressed sequence tags from genes expressed in human leukocytes or having approximately 40,000 expressed sequence tags from genes expressed in a variety of human tissues. In some cases, a variety of additional validation experiments were conducted. As is shown in Table 1, infra, and discussed in detail hereinbelow, several differentially expressed genes were identified. Based, in part, on the identification of these IRM genes, the present invention provides methods and reagents useful for designing and performing diagnostic and prognostic assays for insulin resistance and related conditions; evaluation of risks for diseases such as insulin resistance and related conditions; designing prophylactic and therapeutic regimes for diseases such as insulin resistance and related conditions; screening for agents useful for treatment of diseases such as insulin resistance and related conditions.

As used herein, the term "insulin resistance" has the meaning normally accepted in the art and refers to the resistance of peripheral tissue to the action of insulin to stimulate glucose uptake. If the pancreas is capable of secreting more insulin in response to this defect, normal glucose tolerance can be maintained. Certain abnormalities including hypertension and dislipidemia characterized by increased plasma triglyceride (TG), decreased high-density lipoproteins (HDL), smaller and denser LDL particles, an increase in post prandial lipemia, hyperuricemia, and increased plasminogen activator inhibitor-1 (PAI-1) levels, tend to cluster in hyperinsulinimic patients with insulin resistance are referred to as IR-related conditions. Insulin resistance-related conditions include diabetes (e.g., type II and gestational) and symptoms and complications of diabetes such as Syndrome X (e.g., including reduced levels of circulating high-density lipoproteins, hypertension, abdominal obesity and coronary artery disease) and the like.

III.INSULIN RESISTANT MARKERS As noted supra, the present inventors have identified a panel of genes differentially expressed in cells (e.g., blood cells) of insulin resistant subjects compared to healthy subjects. These genes, referred to as Insulin Resistance Marker, or IRM, genes encode RNAs (i.e., IRM gene products) that hybridize under stringent conditions to a polynucleotide having a sequence of (i.e., identical to or exactly complementary to) a polynucleotide identified by accession number in Table 1, infra (e.g., expressed sequence tag(s), IMAGE clone insert or cDNA sequence(s) having an accession number(s) shown). In certain embodiments, the accession sequence is a genomic sequence. For example, transcripts of IRM genes may hybridize to, for example, (1) a polynucleotide of having an accession sequence of Table 1 or its complement (excluding any poly(A) tail) as well as to (2) a polynucleotide having the sequence of the insert of an IMAGE clone listed in Table 1.

Table 1 provides a variety of types of information. Column 1 provides a numerical designation for each IRM gene. Column 2 shows the GenBank accession number of the EST sequence to which differential hybridization was observed using RNA from eIR and eIS populations as described in § II, supra, and in the Examples, infra. Column 2 also provides the GenBank accession number(s) of longer genomic or cDNA sequences corresponding to certain expressed sequence tags. Column 2 also shows the IMAGE clone ID number corresponding to each EST sequence. IMAGE clones generally contain inserts of from about 1 kb to full-length. The clones are available from Research Genetics, Inc. (http://www.resgen.com/ resources/apps/cdna/ index.php3) and the nucleotide sequences of IMAGE clones can be determined using routine methods.

Column 3 indicates whether the particular IRM is upregulated or downregulated in cell lines of the eIR population compared to the eIS population, as determined as described in the examples, infra. A "" indicates that the IRM gene is downregulated in the eIR population compared to the eIS population (i.e., lower expression in the eIR population). A "+" indicates that the IRM gene is upregulated in the eIR population compared to the eIS population.

Column 4 of Table 1 provides information concerning the full-length gene corresponding to the EST sequence (e.g., typically >95% sequence identity) and/or describes a polypeptide encoded by the gene. Polypeptide sequences encoded by the IRMs are identified in column 4 or in the GenBank annotation accompanying the noted accession number or, alternatively can be determined by conceptual translation of the IRM nucleic acid sequences provided or determinable from the nucleic acid sequence information provided. For convenience, a polypeptide encoded by an IRM gene, or subsequence thereof, is sometimes referred to as an "IRM polypeptide."Additional clones and sequence information (for example, coding sequence, full-length sequence, flanking sequence, genomic sequences) corresponding to the IRMs described herein can be obtained using techniques well known to molecular biologists. For example, the IMAGE clones listed in Table 1 can be obtained and the clone inserts sequenced. Additional clones that may be sequenced are obtained by screening mammalian (e.g., human) cDNA libraries (e.g. blood cell libraries, e.g., lymphocyte cDNA libraries) or genomic libraries using labeled probes having a IRM sequence provided herein. Alternatively, computerized sequence databases can be searched for substantial sequence identity with an accession sequence, subsequences thereof, or polypeptide sequences encoded therein.

IRM genes encode RNAs (IRM RNAs) that hybridize (e.g., under stringent conditions) to a polynucleotide having the sequence of, or exactly complementary to, a sequence identified in Table 1 by GenBank accession number. IRM gene products also include polypeptides encoded by an RNA that hybridizes under stringent conditions to a polynucleotide having the sequence of, or exactly complementary to, an accession sequence. The IRM gene products identified by the inventors comprise a sequence of, or a sequence encoded by, a nucleic acid sequence provided in Table 1, a fragment thereof, or the complement of such a sequence. Polynuclotide probes and primers that specifically hybridize to the IRM sequences (including the complements of sequences) disclosed herein (e.g., in Table 1) can be used to monitor, detect and measure expression of the gene encoding the IRM. For example, typically, the probe contains at least 10 bases identical to, or exactly complementary to, a polynucleotide referred to in Table 1, often at least about 15 bases, at least about 20 bases, at least about 25 bases, at least about 50 bases, at least about 100, or at least about 500 bases. However, in determining sequence identity, complementarity or hybridization, any 3" terminal poly(A) sequence (e.g., provided in cDNA-derived sequences) is not included. In a different embodiment, agents (such as antibodies) that bind polypeptides encoded by the IRM genes can be used to monitor, detect and measure expression of the gene encoding the IRM.

The correlation demonstrated between expression of the IRM genes listed in Table 1 and insulin resistance indicates that expression of the IRM genes is diagnostic of the development of, or likelihood of developing, IR or a related condition. Thus, detection of a change in expression of an IRM RNA that hybridizes to, or has substantial sequence identity with a polynucleotide of an accession sequencedenoted in Table 1, or its complement (including a polynucleotide having the sequence of the insert of an IMAGE clone listed in Table 1) is useful in the diagnostic, prognostic and screening methods of the invention. Similarly, a change in the expression or activity of a polypeptide that is encoded by an IRM gene (and/or a polypeptide encoded by an IRM gene), is useful in the diagnostic, prognostic and screening methods of the invention, as described below.

The correlation demonstrated between expression of the IRM genes comprising a sequence provided in Table 1 and insulin resistance similarly indicates that the IRM genes likely have a causative role in the manifestation of IR. Accordingly, the present disclosure provides methods of treating IR by administering an agent or treatment that modulates expression of an IRM protein that is encoded by an RNA that hybridizes (e.g., under stringent conditions) to any of polynucleotides disclosed herein. Numerous other aspects and embodiments of the invention are described herein or will be apparent upon review of the disclosure.

| | | | |
|---|---|---|---|
| 1 | 2 | 4 | 5 |
| IRM No. | EST Acc. No.IMAGE ID #GenBankAcc. No. | Relative Expression | Polypeptide encoded by IRM geneComments |
| IRM1 | AA971714 1584588 M87320 | + | Homo sapiens clone BCSynL38 immunoglobulin lambda light chain variable region mRNA, partial cds |
| IRM4 | AA962431 1553550 AK055867 | + | cDNA FLJ31305 moderately similar to Rattus norvegicus kidney-specific protein (KS) mRNA |
| IRM9 | AI820640 1604668 | - | Epsilon-tubulin |
| IRM10 | H22559 51807 NM_025135.1 AB051482.1 | - | Hypothetical Protein FLJ22297 (KIAA1695) |
| IRM11 | AI146565 1703053 NM_006681 | - | Neuromedin U |
| IRM12 | N79432 288827 AK000972 | + | Hypothetical protein FLJ10110 |
| IRM16 | H97646 250328 AK022892 | + | Homo sapiens cDNA FLJ12830 |
| IRM18 | W07745 300972 | - | Hypothetical protein BC010734 |
| IRM19 | AA598865 897963 XM_042108 | - | KIAA0052 protein |
| IRM20 | R26131 133085 BC007351.1 XM_041375.3 | - | Hypothetical Protein FLJ22297 |
| IRM21 | T74394 84560 NM_022748 | - | Tumor endothelial marker 6 |
| IRM25 | AA464464 810448 AK024224 | + | Homo sapiens cDNA FLJ14162 |
| IRM27 | R99831 201045 | + | KIAA1034 protein |
| IRM28 | AA487700 841641 NM_053056 | - | Cyclin D1 |
| IRM29 | R28669 133895 HSA420583 | + | Homo sapiens mRNA full length insert cDNA clone |
| IRM30 | AA005202 429083 | + | Expressed sequenced tag; contained in BAC Accession # AL356216 |
| IRM33 | AI299994 1909455 S72730 | + | Homo sapiens isolate donor D clone D105K immunoglobulin kappa light chain variable region mRNA |
| IRM40 | AA625979 745490 XM_006697.3 NM_017899.1 | - | Hypothetical protein FLJ20607 |
| IRM44 | H08397 45501 | + | Ubiquitin carboxyl-terminal esterase L1 (ubiquitin thiolesterase) |
| IRM49 | AI792160 1634992 BC025747 | + | Homo sapiens, similar to solute carrier family 25 (carnitine/acylcarnitine translocase), member 20, mRNA |
| IRM50 | AA418544 767313 | - | Human homolog of mouse nuclear receptor - subfamily 2, group F, member 2 (Nr2f2) |
| IRM51 | AA047418 488130 AK000774 | + | Homo sapiens cDNA FLJ20767 |
| IRM52 | W01830 298134 NM_003505 | + | Frizzled homolog 1 (Drosophila) |
| IRM56 | AI192675 1743572 NM_007369 | - | G-protein coupled receptor |
| IRM57 | AA936866 1486194 AF001862 | + | FYN binding protein (FYB-120/130) |
| IRM60 | AA453769 813697 AB018289.1 XM_045277.3 | - | Hypothetical protein KIAA0746 |
| IRM62 | AA625673 745367 NM_139163 | + | Homo sapiens ALS2CR12 mRNA |
| IRM66 | R72517 156043 AK025586 | + | Homo sapiens cDNA: FLJ21933 |
| IRM67 | H99427 262264 NM_002845 | + | Protein tyrosine phosphatase, receptor type, M |
| IRM68 | AA504392 825234 | + | Hypothetical protein DKFZp762M186 |
| IRM69 | R67000 140337 | + | Pregnancy-associated plasma protein A |
| IRM70 | AA917071 1526555 | + | EST |
| IRM73 | AA427970 773469 XM_040709 | + | Prostaglandin F2 receptor negative regulator |
| IRM74 | AI369629 2017415 NM_001809 | + | Centromere protein A (17kD) |
| IRM75 | W93178 357084 | + | HSPC125 protein |
| IRM77 | AA463792 796508 NM_015179 | + | KIAA0690 protein |
| IRM78 | AA608576 950689 NM_014268 | + | Microtubule-associated protein, RP/EB family, member 2 |
| IRM80 | 1631355 XM_028959 | - | LASP-1, LIM and SH3 protein 1 |
| IRM81 | AA485365 811010 | + | Homo sapiens, clone MGC: 4710 |
| IRM84 | AA923509 1534589 AF368463 | + | Carboxypeptidase M |
| IRM85 | AA778890 453289 AK000103 | + | Homo sapiens cDNA FLJ20096 |
| IRM90 | AI017655 1635933 BC002677.1 | + | Hypothetical protein DJ159A19.3 |
| IRM92 | H41574 175767 AB007979 | + | Homo sapiens mRNA, chromosome 1 specific transcript KIAA0510 |
| IRM94 | AA099593 489722 NM_014900 | + | KIAA0977 protein |
| IRM100 | AI299601 1900149 AF077599.1 | + | Hypothetical protein SBBI03 |
| IRM110 | AI350226 1910316 NM_014682.1 | + | KIAA0535 gene productNagase, et.al. DNA Res 5: 31 (1998) |
| IRM118 | H17022 50781 AF396687 | + | Homo sapiens rab effector MYRIP (MYRIP) mRNA |
| IRM119 | AI189606 1725451 NM_002288 | - | Leukocyte-associated Ig-like receptor 2 |
| IRM120 | AA055136 377384 M64497.1 | - | Apoprotein AI regulatory protein (ARP-1) |
| IRM122 | AA458779 838366 NM_000191 | - | 3-hydroxymethyl-3-methylglutaryl-Coenzyme A lyase (hydroxymethylglutaricaciduria) |
| IRM124 | AA485055 815871 NM_012443 | - | Sperm associated antigen 6 |
| IRM130 | AA465345 814057 AA465345 | - | EST |
| IRM136 | AA194833 664975 NM_021101 | - | Claudin 1 |
| IRM140 | AA486321 840511 BC000163.2 AK056766.1 | - | Vimentin |
| IRM146 | AA458934 814432 | - | Hypothetical protein AF301222 |
| IRM148 | N57005 277589 NM_005977 | + | Ring finger protein (C3H2C3 type) 6 |
| IRM150 | AA488341 842994 AF136273.1 NM_001336.1 | + | Cathepsin Z (CTSZ) |
| IRM152 | AA452431 786590 NM_004967 | + | Integrin-binding sialoprotein (bone sialoprotein, bone sialoprotein II) |
| IRM160 | AA598840 898328 XM_018136.1 | + | Early development regulator 2 (homolog of polyhomeotic 2) |
| IRM165 | AA827405 1422794 | + | Mucosa associated lymphoid tissue lymphoma translocation gene 1 |
| IRM170 | AI300810 1901363 AJ000673.1 NM_007334.1 | + | CD94 protein, c-type lectin |
| IRM178 | W47366 324719 | - | Mitochondrial ribosomal protein L39 |
| IRM180 | AA485739 811139 BC007920.1 | - | HLA class II, DR-1 beta chain |
| IRM182 | AA426066 757236 XM_087410 | - | Hypothetical protein BC007882 |
| IRM188 | AA188528 625933 NM_032299 | - | Hypothetical protein MGC2714 |
| IRM190 | AA971714 1584588 M87320.1 | + | IG lambda light chain precursor V-VI regionStephen Nuc Acid Res 25:3389 (1997) |
| IRM200 | AI299994 1909455 X58082.1 | + | IG kappa chain precursor V-III regionStephen Nuc Acid Res 25:3389 (1997) |
| IRM202 | AA521337 826138 NM_000156 | - | Guanidinoacetate N-methyltransferase |
| IRM207 | AI202954 1942549 XM_052415 | - | Calcium channel, voltage-dependent, L type, alpha 1C subunit |
| IRM210 | W72870 344959 XM_003392.2 NM_018401.1 | - | Serine/threonine protein kinase |
| IRM217 | AA043997 486984 BC007523 | - | Hypothetical protein MGC14961 |
| IRM220 | AA417274 731203 BC005839.1 | - | Follistatin-like 3 (secreted glycoprotein) |
| IRM228 | N32593 259951 NM_001623 | - | Allograft inflammatory factor 1 |
| IRM230 | AA505045 825648 X58399.1 XM_034917.1 | - | L2-9 transcript of unrearranged IG V (H) 5 pseudogeneBerman J Exp Med 173: 1529 (1991) |
| IRM236 | T65861 81599 NM_005151 | - | Ubiquitin specific protease 14 (tRNA-guanine transglycosylase) |
| IRM240 | AA857944 1435624 AA857944 | + | Homolog of mouse proteoglycan PG-M isoform mRNAShinomrua JBC 270: 0328 (1995) |
| IRM244 | AI147534 1555659 NM_002084 | - | Glutathione peroxidase 3 (plasma) |
| IRM248 | AI091722 1651147 NM_002977 | + | Sodium channel, voltage-gated, type IX, alpha polypeptide |
| IRM250 | R08117 127099 AK027735.1 XM_034690.3 | - | FLJ14829 cDNA; contains PDZ domain |
| IRM255 | AI095381 1666549 NM_002232 | - | Potassium voltage-gated channel, shaker-related subfamily, member 3 |
| IRM259 | AA977181 1587374 AK056644 | + | Homo sapiens cDNA FLJ32082 |
| IRM260 | AA779727 1034494 Y13786.2 NM_033274.1 | + | Meltrin-beta/ADAM 19 homolog |
| IRM266 | N31244 265494 NM_080927 | - | Endothelial and smooth muscle cell-derived neuropilin-like protein |
| IRM270 | AA903183 1517171 XM_005707.1 | + | Interleukin 2 receptor alpha |
| IRM277 | T59043 74537 NM_001134 | + | Alpha-fetoprotein |
| IRM278 | R56202 41004 | + | Myelin transcription factor 1-like |
| IRM280 | AA889789 1460828 XM_005116.3 NM_004103.2 | - | TRPM, nicotinic acetylcholine receptorProtein tyrosine kinase of focal adhesion kinase subfamily |
| IRM288 | AA995045 1631546 | - | Melanoma antigen, family A, 3 |
| IRM290 | T70057 80948 M12759.1 XM_059628.2 | + | Ig J chain |
| IRM296 | N25141 261494 | + | Cullin 3 |
| IRM297 | AA011465 429555 | + | Fibrinogen, A alpha polypeptide |
| IRM300 | AA055768 510576 AF038452.1 | + | Secreted cement gland protein XAG-2 homolog (hAG-2/I)Thompson BBRC 251: 111 (1998) |
| IRM303 | W05003 295412 | - | EST |
| IRM309 | AA400893 727792 | - | Phosphodiesterase 1A, calmodulin-dependent |
| IRM310 | AA421515 739116 AF136273.1 | + | Cathepsin Z (CTSZ) |
| IRM314 | AA043772 486401 | - | Hypothetical protein BC006258 |
| IRM320 | AI299075 1900284 U11552.1 | + | Leukotriene-C4 synthetaseWelsch PNAS 91: 9745 (1994) |
| IRM326 | AA460093 796461 | + | General transcription factor IIIA |
| IRM328 | R34323 136449 | + | Hypothetical protein FLJ10357 |
| IRM330 | AI278730 1911864 NM_004485.1 XM_084057.4 | + | G protein gamma-4Ray et.al. JBC 15: 1765 (1995) |
| IRM331 | AA464062 810272 | - | Protein phosphatase 1, regulatory (inhibitor) subunit 12B |
| IRM332 | AA479326 753610 | + | Apolipoprotein E |
| IRM336 | AI022884 1650660 | + | Synaptotagmin XII |
| IRM340 | A1091722 1651147 M94055.1 NM_002977.1 | + | Human voltage-gated sodium channel proteinAhmed CM et.al. PNAS 89: 8220-8224 (1992) |
| IRM344 | AA018655 362732 | + | Hypothetical protein BC012365 |
| IRM350 | AA885871 1500420 | + | EST |
| IRM351 | 470393 NM_002423 | + | Homo sapiens matrix metalloproteinase 7 (matrilysin, uterine) (MMP7) |
| IRM352 | AA669443 884867 NM_001969 | + | Eukaryotic translation initiation factor 5 (EIF5) |
| IRM353 | AA875913 1492202 | + | EST |
| IRM354 | H88540 253009 BC025986 | + | Similar to cyclic nucleotide gated channel, cGMP gated |
| IRM355 | AA232417 664233 NM_000848 | + | Glutathione S-transferase M2 (muscle) (GSTM2) |
| IRM356 | N57849 247084 | - | EST |
| IRM357 | AA151413 504742 | - | EST |
| IRM358 | H92779 231944 | - | EST |
| IRM359 | AA418545 767315 NM_005481 | - | Thyroid hormone receptor-associated protein, 95-kD subunit (TRAP95) |
| IRM360 | W69816 343923 NM_139247 | - | Adenylate cyclase 4 (ADCY4) |
| IRM361 | AI420444 2095501 NM_023076 | - | Hypothetical protein FLJ23360 (FLJ23360), |
| IRM362 | AA946732 1421061 XM_037206 | - | Homo sapiens GTP binding protein 5 (putative) (GTPBP5) |
| IRM363 | AI824220 2404902 NM_005026 | - | Homo sapiens phosphoinositide-3-kinase, catalytic, delta polypeptide (PIK3CD) |

IRM polynucleotides and polypeptides (e.g., for use as probes, immunogens, and the like) can be obtained using methods well known in the art, including de novo chemical synthesis and recombinant expression. Methods for de novo synthesis of oligo and polynucleotides are known (see, Beaucage et al. eds., Current Protocols in Nucleic Acid Chemistry John Wiley & Sons, Inc., New York, 2000); and Agrawal, ed., Protocols for Oligonucleotides and Analogs, Synthesis and Properties Humana Press Inc., New Jersey, 1993). Examples of solid-state methodologies for synthesizing proteins are described by Grant (1992) Synthetic Peptides: A User Guide, W.H. Freeman and Co., N.Y.; and in Principles of Peptide Synthesis, (Bodansky and Trost, ed.), Springer-Verlag, Inc. N.Y., (1993).

Methods for recombinant expression of polynucleotides and polypeptides are well known in the art. For example, the IRM polynucleotides can be inserted into expression vectors for the preparation of IRM polypeptides and polynucleotides. Expression vectors typically include transcriptional and/or translational control signals (e.g., transcriptional regulatory element, promoter, ribosome-binding site, and ATG initiation codon). In addition, the efficiency of expression can be enhanced by the inclusion of enhancers appropriate to the cell system in use. For example, the SV40 enhancer or CMV enhancer can be used to increase expression in mammalian host cells. Thus, in one embodiment, DNA encoding an IRM polypeptide is inserted into DNA constructs capable of introduction into and expression in an in vitro host cell, such as a bacterial (e.g., E. coli, Bacillus subtilus), yeast (e.g., Saccharomyces), insect (e.g., Spodoptera frugiperda), or mammalian cell culture systems. Examples of mammalian cell culture systems useful for expression and production of the polypeptides of the present invention include human embryonic kidney line (293; Graham et al., 1977, J. Gen. Virol. 36:59); CHO (ATCC CCL 61 and CRL 9618); human cervical carcinoma cells (HeLa, ATCC CCL 2); and others known in the art. Useful human and nonhuman cell lines are widely available, e.g., from the American Type Culture Collection (ATCC), P.O. Box 1549, Manassas, VA 20108 (see http://www.atcc.org). The use of mammalian tissue cell culture to express polypeptides is discussed generally in Sambrook, supra, and Ausubel, supra.

In some embodiments, promoters from mammalian genes or from mammalian viruses are used, e.g., for expression in mammalian cell lines. Suitable promoters can be constitutive, cell type-specific, stage-specific, and/or modulatable or regulatable (e.g., by hormones such as glucocorticoids). Useful promoters include, but are not limited to, the metallothionein promoter, the constitutive adenovirus major late promoter, the dexamethasone-inducible MMTV promoter, the SV40 promoter, and promoter-enhancer combinations known in the art.

IRM polypeptides or fragments can also be expressed in transgenic animals (mouse, sheep, cow, etc.) and plants (tobacco, arabidopsis, etc.) using appropriate expression vectors which integrate into the host cell chromosome.

IV.DIAGNOSTIC AND PROGNOSTIC METHODSIn one aspect, the invention provides a means for determining if a subject has, or is at risk of developing, insulin resistance or related conditions that are associated a change in the expression profile of one or more IRM genes. In one aspect of the invention, the expression of an IRM gene product is monitored for diagnosis of individuals susceptible to, or suffering from, insulin resistance and related conditions. In a related aspect, IRM expression is monitored for prognostic evaluations to detect individuals at risk for insulin resistance and related conditions. Prognostic methods can also be utilized in the assessment of the severity of the disease and appropriate methods of treatment. Assays for the presence or quantity (absolute or relative) of IRM gene products may be carried out and the results interpreted in a variety of ways, depending on the assay format, the nature of the sample being assayed, and the information sought.

Thus, in one aspect, the invention provides a method for diagnosing for insulin resistance (IR), IR-related conditions, or susceptibility to IR or IR-related conditions in a subject by detecting a difference in expression of at least one insulin resistance marker (IRM) listed in Table 1 in a biological sample from the subject compared to the level of expression of the IRM characteristic of expression in a similar biological sample in a reference population of individuals who are not insulin resistant (e.g., a population of individuals with an eIS phenotype). The reference population may be gender, age and/or ethnicity matched to the subject. In an embodiment, the level of expression of the IRM is determined by detecting an IRM RNA, for example by hybridizing a probe derived from RNA of the subject to an immobilized polynucleotide target, and detecting the formation of a hybridization complex. Useful targets include polynucleotides that hybridize to an IRM gene listed in Table 1. Suitable probes include optionally labeled cDNA probes prepared using RNA from the subject, optionally labeled RNA isolated from the subject, optionally labeled amplification products or RNA or cDNA, or other detection probes (e.g., so-called invader-directed cleavage, e.g. US Pat. No. 6,001,567). In an embodiment, the probe is hybridized to an array of immobilized polynucleotides, wherein said immobilized polynucleotides comprise polynucleotides that hybridize to at least two different IRM genes listed in Table 1.

Based on a diagnosis of insulin resistance, a physician can provide appropriate medical treatment and advice to ameliorate the symptoms or effects of the condition, or to return the patient to a non-insulin resistant status. Although susceptibility to insulin resistance has historically been determined by taking a family history of diabetes, hypertension, obesity, results of OGTT and SSPG tests, and other known risk factors known to those of skill (such as low HDL, high triglycerides, and the like) the present invention provides additional methods for identifying patients with high susceptibility to insulin resistance (i.e., with greater susceptibility than average in the general population, i.e., at high or above-average risk). Patients identified as susceptible can be afforded prophalactic treatments to avoid development or worsening of the condition.

The invention provides a method for diagnosing for insulin resistance or susceptibility to developing insulin resistance in a patient by determining the level of expression of an IRM gene in a tissue sample from the patient and comparing the level of IRM gene expression to expression levels characteristic of a population with a known insulin resistance status, such as subjects who are not insulin resistant. The level (e.g., average level) of IRM gene expression in a population of subjects who are not insulin resistant and/or not considered at high risk for developing insulin resistance (a "healthy"population, e.g., the eIS population) is referred to as the "normal" level. A difference in the level of IRM gene expression is indicative of a diagnosis of insulin resistance or susceptibility to insulin resistance. The difference can be a decrease or an increase relative to normal levels. In some embodiments, the diagnostic and prognostic methods of the invention involve obtaining a biological sample, usually a tissue sample, preferably a blood sample, from a subject. Samples used for detection of IRM gene expression and other diagnostic methods of the invention can be obtained from a variety of sources. Since the methods are designed primarily to diagnosis and assess risk factors for humans to insulin resistance and related conditions (e.g., Type II diabetes) samples are typically obtained from a human subject. However, the methods can also be utilized with samples obtained from other mammals, such as non-human primates (e.g., apes and chimpanzees), mice and rats, or from in vitro cell cultures, for example to conduct drug screening assays and/or preclinical toxicity and efficacy tests. Such samples can be referred to as a "biological sample." Biological samples useful in the practice of the invention include a blood sample, serum, cells (including whole cells, cell fractions, cell extracts, and cultured cells or cell lines), tissues (including tissues obtained by biopsy), cells from body fluids (e.g., urine, sputum, amniotic fluid, synovial fluid, semen, saliva, tears, spinal fluid), or cultured cells or cell lines. A biological sample obtained from a patient is sometimes referred to herein as a "patient sample." The biological sample can, of course, be subjected to a variety of well-known post-collection preparative and storage techniques (e.g. storage, freezing, etc.) prior to assessing the amount of the IRM gene product in the sample.

In one embodiment, the biological samples are blood or a blood component from a patient. For example, blood can be collected following an 8-hour fast by draw into a evacuated tube (e.g. "vacutainer" blood collection tubes) containing, for example, disodium ethylenediamine-tetracetic acid (EDTA) at 1.5 mg/ml of blood. If desired, leukocytes are collected by centrifugation at 1500xg for 30 minutes, at 4ºC, within 2 hours of blood collection. The interface between the top plasma layer and the bottom red blood cell layer containing white blood cells (buffy coat), is collected for analysis (e.g., RNA extraction using standard methods).

The level of expression of an IRM gene in a tissue sample from the patient can be compared to normal levels expression levels in a population with a known insulin resistance status (e.g., healthy subjects) in a number of ways. For example, the level of IRM gene expression in the tissue sample is compared to a reference or baseline value. Although the reference value can be the level of expression of an IRM in an individual of known insulin resitance status, generally the reference value is the level of expression of the IRM characteristic of expression of a population (i.e., a reference population) of individuals of known insulin status (e.g., eIS phenotype, eIR phenotype, etc.). As discussed below, usually, the reference value is a statistical value (e.g., a mean or average) established from a population of at least 3, and usually at least 5 or more individuals. A reference hereinbelow to a value characteristic of an individual will be understood to also refer to a value characteristic of a population of individuals.

As described below, typically the reference or baseline value is a level of IRM expression characteristic of a healthy subject. Examples of healthy subjects include individuals not suffering from IR or, in some embodiments, not at high risk for developing IR, including, in some embodiments, subjects or populations with an eIS phenotype). A difference between the experimental or determined level measured in the subject (i.e., a "test value") and the reference value is an indication that the subject suffers from, or is at risk for developing, insulin resistance or a related condition.

For purposes of diagnosis, the reference value can be the level of IRM gene expression in a healthy subject. Alternatively, the reference value can be the level of IRM expression in a tissue sample from the test subject that is obtained at earlier or later time. Usually, the reference value is a statistical value (e.g., a mean or average) established from a population of control cells or individuals. The population that serves as a control can vary in size, having as few as a single member, but potentially including tens, hundreds, or thousands of individuals. Usually the reference values are determined based on a population size of at least 3 individuals, or optionally at least 5 individuals, in each population. When the control is a large population, the reference value can be a statistical value determined from individual values for each member or a value determined from the control population as an aggregate (e.g., a value measured for a population of cells within a well). Thus, for instance, the reference value can be a statistical level or range that is reflective of IRM levels for the general population, more usually healthy individuals not suffering from and not at increased risk for IR, and in some cases a population of individuals with an eIS phenotype.

For purposes of determining reference or baseline values, a healthy individual (i.e., an individual not suffering from IR) can be identified by the following criteria: fasting glucose < 95 mg/dl, 75-g 2-hr OGTT glucose <120mg/dl and preferably <100 mg/dl, SSPG mean <150 mg/dl and preferably <120 mg/dl. 75-g 1hr or 2-hr insulin < 60 µ IU/ml. Individuals with an eIS phenotype (who are also healthy individuals) also can be used for establishing a baseline or reference value. The criteria for identifying individuals with the eIS phenotype are provided above. Insulin resistance can also be determined using the euglycemic insulin clamp technique (Andres et al., 1966, in Automation in Analytical Chemistry; Skeggs LT Ed. P.486-91) and the minimal model (Bergman et al., 1987, J Clin Invest 79:790-800).

Normal levels of IRM expression can be determined for any particular population, subpopulation, or group of organisms according to standard methods well known to those of skill in the art. Application of standard statistical methods permits determination of baseline levels of expression, as well as identification of significant deviations from such reference values. Thus, for example, the levels of IRM expression in a population (e.g., at least 3, at least 5 or at least 10 individuals) can be determined and routine methods can be used to define a statistically significant difference from the population. A difference is typically considered "statistically significant"if the probability of the observed difference occurring by chance (the p-value) is less than some predetermined level. As used herein a "statistically significant difference" refers to a p-value that is < 0.05, preferably < 0.01 and most preferably < 0.001.

The magnitude of the difference in expression of IRM genes in subjects that are insulin resistant or have increased susceptibility to insulin resistance compared to a population of healthy individuals will vary depending on the gene and severity of the condition. In some embodiments, expression of an IRM gene in a test subject is considered different (upregulated) compared to a reference value when the test value is at least about 25% higher than the reference value, often at least about 50% higher, sometimes increased by 50 to 100%, in other instances from about 2- to about 5-fold higher or any integer therebetween (i.e., 3-fold or 4-fold), in still other instances between about 5- and about 10-fold higher or any integer therebetween, sometimes between about 10- and about 20-fold higher or any integer therebetween, in other instances between about 20- and about 50-fold higher or any integer therebetween, in yet other instances between about 50- and about 100-fold or higher or any integer therebetween, and in still other instances 100-fold higher or more. In some embodiments, expression of an IRM gene in a test subject is considered different (downregulated) compared to a reference value when the test value is at least about 25% lower than the reference value, often at least reduced about 50% lower, sometimes reduced by 2- to about 5-fold or any integer therebetween, in still other instances by between about 5- and about 10-fold or any integer therebetween, sometimes between about 10- and about 20-fold or any integer therebetween, in other instances between about 20- and about 50-fold or any integer therebetween, in yet other instances between about 50- and about 100or any integer therebetween, and in still other instances 100-fold or more.

In some embodiments, levels of IRM protein or IRM mRNA are determined by quantitating the amount of IRM protein and/or mRNA in biological samples obtained from subjects, e.g., a human subject. However, it will be appreciated that the assay methods do not necessarily require measurement of absolute values of IRM expression, unless it is so desired, because relative values are sufficient for many applications of the methods of the present invention. Where quantitation is desirable, the present invention provides reagents such that virtually any known method for quantitating gene products can be used.

Because IRM expression levels may vary from tissue to tissue, the test value and the reference or baseline value are preferably determined from the same tissue (e.g., blood or a specified blood fraction, e.g., B-lymphocytes, T-lymphocytes, monocytes, neutrophils, or other white blood cells). For certain samples and purposes, one may desire to quantitate the amount of IRM gene product on a per cell, or per volume, basis. In addition, it will be recognized that it is generally desirable that the test values and reference values are obtained under similar conditions. For example, when a blood sample is used, typically the blood will be collected under fasting conditions (i.e., no caloric intake for at least 8 hours, e.g., by an overnight fast).

In one embodiment, for example, to assess insulin resistance, data are collected to obtain a statistically significant correlation of disease severity or progression with different IRM expression patterns and a predetermined range of IRM levels is established for the same cell or tissue sample obtained from subjects having known clinical outcomes. A sufficient number of measurements is made to produce a statistically significant value (or range of values) to which a comparison will be made. The predetermined range of IRM levels or activity for a given cell or tissue sample can then be used to determine a value or range for the level of IRM gene product that would correlate to favorable (or unfavorable) prognosis. The level of IRM gene product from a biological sample (e.g., a patient sample) can then be determined and compared to the low and high ranges and used to predict a clinical outcome.

In carrying out the diagnostic and prognostic methods of the invention, as described above, it will sometimes be useful to refer to "diagnostic"and "prognostic values."As used herein, "diagnostic value" refers to a value that is determined for the IRM gene product detected in a sample which, when compared to a normal (or "baseline") range of the IRM gene product is indicative of the presence of a disease (e.g., insulin resistance or Type II diabetes). "Prognostic value"refers to an amount of the IRM gene product detected in a given cell type (e.g., blood cell) that is consistent with a particular diagnosis and prognosis for the disease. The amount (including a zero amount) of the IRM gene product detected in a sample is compared to the prognostic value for the cell such that the relative comparison of the values indicates the presence of disease or the likely outcome of the disease progression.

In some embodiments of the invention, the subject is identified as a patient at risk, or at increased risk, for insulin resistance prior to, or after, conducting the assay. For example, a subject can be identified as at risk based the medical history of the subject or the subject"s family.

Diagnosis of IR and related conditions can also be based on the detection of polymorphism in the IRM genes in the biological sample from the subject, as is discussed in greater detail below. Thus, in an aspect of the present invention, assays of the sequence (i.e., polymorphisms) or expression of IRM genes are used to identify individuals more likely to develop insulin resistance than the population average. In one embodiment, the invention provides a method of determining whether an individual is insulin resistant by identifying a patient at risk for IR (or suspected of being at risk), obtaining a tissue sample of an individual and comparing the level of IRM expression in the tissue sample to a reference value.

It will recognized by the reader that the methods described herein can also be used (with modifications that will be apparent) to diagnose or screen for individuals with an extreme insulin sensitivity phenotype.

Assays for Expression of Panels of IRMsIn some embodiments, the invention provides diagnostic, pronostic, and drug screening assays (e.g., as described below) in which the expression level of more than one IRM gene ("a panel of IRM genes") is monitored. These methods are also useful for monitoring the progression of IR-related conditions and the effectiveness of treatment. Monitoring expression of multiple genes provides for more robust assays.

Thus, in various embodiments, gene expression profiles encompassing a combination of IRM genes (e.g., at least 2, 3, 4, 5, 6 7, 8, 9, 10, 11, 12, 15, 20, or 25 or more of the genes listed in Tableor in a subpanel thereof) are determined for a subject (e.g., for diagnostic and prognostic assays) or cell line (e.g., for drug screening assays). Expression levels can be determined by any of a number of methods for detecting RNA or protein levels (e.g., membrane or microarray hybridization, RT PCR, and the like) including without limitation the methods described infra. Devices comprising arrays of probes for specific IRM gene products, e.g., as described herein, may be used to conduct the assays.

Useful subpanels of IRM genes can be selected based structural, functional or other criteria. Examplary panels include, without limitation, panels comprising, e.g., (a) IRM 1, 21, 33, 124, 180, 190, 200, 230, 288, and 290; (b) IRM 11, 44, 84, 122, 136, 140, 150, 202, 210, 236, 244, 296, 309, 310, 320, and 336; (c) IRM 50, 56, 67, 119, 170, 270, and 280; (d) IRM 6, 10, 11, 28, 56, 57, 67, 73, 80, 118, 120, 148, 152, 160, 170, 178, 207, 210, 228, 248, 250, 255, 270, 280, 330, 331, 332, and 340; (e) IRM 110, 278, and 326; (f) IRM 6, 74, 78, 266, and 297; (g) IRM 4, 12, 16, 18, 19, 20, 25, 27, 29, 40, 49, 51, 52, 60, 62, 66, 68, 75, 77, 85, 90, 92, 94, 100, 146, 182, 188, 217, 240, 250, 259, 260, 314, 328, and 344; (h) IRM 69, 220, 228, 244, 277, and 300; (i) IRM 10, 20, 40, 50, 60, 120, 130, 180, 190, 200, 210, 220, and 260; (j) IRM 90, 150, 160, 170, 250, and 300; (k) IRM 30, 70, 81, 130, and 303 (l) IRM 10, 20, 40, 50, 60, 120, 130, 220, and 260; (m) IRM 10, 20, 40, 50, 60, 120, and 130; (n) IRM 10, 20, 40, 50, 60, and 130; (o) IRM 90, 160, 170, 250, and 300; (p) IRM 120; (q) IRM 350, 351, 352, 353, 354, 355, 356, 357, 358, 359, 360, 361, 362, and 363; (r) IRM 350, 352, 353, 354, 355, 356, 357, 358, 359, 360, 361, 362, and 363; (s) IRM 350, 351, 353, 354, 355, 356, 357, 358, 359, 360, 361, and 362 (t) at least 2, 3, 4, 5, 6, 7, 8, 9, or at least 10 insulin resistant markers selected from a panel. As noted, in various embodiments, diagnostic, prognostic, drug screening or other assays may monitor expression (i.e., the gene expression profile) of any combination of IRM genes, such as combinations comprising at least 2, 3, 4, 5, 6, 7, 8, 9, or at least 10 of the insulin resistant markers listed in Table 1 or a subpanel of the insulin resistant markers listed in Table 1.

Assays using all combinations of two or more IRM genes are contemplated by the present invention. In one embodiment the invention provides a method of determining whether an individual is insulin resistant, at risk for developing insulin resistance, or insulin sensitive, by obtaining a biological sample taken from the subject, comparing the expression level IRM genes in the sample to reference values representative of expression in an individual of a known insulin resistance status (e.g., as determined from a population of individuals). Thus, in one embodiment the reference value is characteristic of expression in a subject who is insulin resistant or at risk for developing insulin resistance.

The same methods and reference levels can be used when assaying a panel of several IRM genes as can be used to measure and compare expression of single genes. When a panel of IRM genes is used, the diagnosis can be based on the number and identity of IRM genes whose expression in the subject is similar to a given reference level characteristic of a population of known insulin resistance status (e.g., eIR phenotype). In one embodiment, for example, it is concluded that the individual is insulin resistant or at risk for developing insulin resistance when the expression level of at least 50% (optionally at least 25% or at least 75%) of the IRM genes is similar to reference value characteristic of expression in the insulin resistant or high susceptibility population. In another embodiment, the reference value is characteristic of expression in a healthy subject and it is concluded that the individual is insulin resistant or at risk for developing insulin resistance when the expression level of at least 50% (optionally at least 25% or at least 75%) of the IRM genes is different from a the reference value.

Thus, in one embodiment, the invention provides a method of diagnosing an individual as insulin resistant or at increased risk for developing insulin resistance by obtaining a biological sample taken from the subject, and comparing the expression level of a panel of at least 3 insulin resistance markers listed in Table 1 in the sample to a reference value representative of expression in a population of individuals of a known insulin resistance status, wherein the individual is diagnosed as insulin resistant or at risk for developing insulin resistance when the expression level of at least 50% of the at least 3 insulin resistance markers is not statistically different to reference value, if the reference value is characteristic of expression in a population of subjects who are insulin resistant or the expression level of at least 50% of the at least 3 insulin resistance markers is statistically different from a reference value, if the reference value is characteristic of expression in a population of subjects who are not insulin resistant. In an embodiment, the subjects who are insulin resistant have an eIR phenotype and/or the subjects who are not insulin resistant have an eIS phenotype.

Monitoring Expression of IRM Gene Products

In one aspect of the invention diagnostic and prognostic methods involve detecting expression of an IRM gene product (RNA or polypeptide). Such assays are used in diagnostic, prognostic, drug screening and other applications. In some embodiments, the level of IRM gene expression in a subject or cell is compared to a reference value, as described herein.

Guided by the disclosure herein, it will be apparent to an ordinarily skilled practitioner that any of a variety of methods can be used to detect IRM expression in a qualitative, quantitative or semi-quantitative fashion. For example, IRM gene expression can be monitored by detecting a specified polynucleotide (e.g., an IRM RNA) or a specified polypeptide (e.g., an IRM protein). Suitable methods for detecting a specified polynucleotide include, without limitation, dot blots, Northern blots, in-situ hybridization, hybridization to high-density polynucleotide or oligonucleotide arrays, nucleic acid amplification methods (e.g., quantitative reverse-transcription PCR), RNAase protection methods, and the like. Suitable methods for detecting a specified polypeptide include, without limitation, immunoassays that utilize an antibody or other binding agents that specifically binds to an IRM polypeptide or epitope (e.g., ELISA, Western blots, and the like), or assays for an enzymatic activity indicative of the presence of the IRM polypeptide. For illustration, and not limitation, examples of suitable assays for detection of IRM RNA and polypeptides are discussed below in additional detail.

Assays for IRM PolynucleotidesSome diagnostic and prognostic methods of the invention involve the detection of IRM RNA transcripts in a biological sample. To measure the RNA levels, nucleic acids from, or derived from, the biological sample are obtained. A nucleic acid derived from a biological sample refers to a nucleic acid for whose synthesis a mRNA transcript in the sample (or a subsequence thereof) has ultimately served as a template. For example, a cDNA reverse transcribed from an mRNA, an RNA transcribed from that cDNA, a DNA amplified from the cDNA, an RNA transcribed from the amplified DNA, are all "derived" from the mRNA transcript, and detection of such derived products is indicative of the presence and/or abundance of the original transcript in a sample. Thus, suitable samples include, but are not limited to, mRNA transcripts of IRM, cDNA reverse transcribed from the mRNA, cRNA transcribed from the cDNA, DNA amplified from IRM nucleic acids, and RNA transcribed from amplified DNA. In some embodiments, these methods begin with the lysis of cells and subsequent purification of nucleic acids from other cellular material. However, it is generally not necessary that purification of nucleic acids from other materials be complete. RNA is obtained from a biological sample from a subject using any of a variety of techniques known in the art (see Sambrook and Ausubel, supra).

For example, when beginning with a blood sample from a fasting subject, RNA is collected , red blood cells are lysed and white blood cells are collected which in turn are lysed by adding 1.3 ml TRIZOL (Life Technology Incorporation, GIBCOBRL, Cat# 15596-018) per 10 ml of whole blood. 300 microliters chloroform is added to trigger the phases separation process of the mixture, followed by vigorous shaking and a period of standing. Centrifugation at 12,000rpm for 15 minutes is performed to completely separate the mixture into aqueous phase containing RNA, and organic phase, which contains genomic DNA and protein. The aqueous phase is collected and RNA prepared by ethanol precipitation. The integrity and quantity of the purified RNA can be determined using side-by-side gel electrophoresis (1% agarose gel in electrophoresis tank containing 0.1% DEPC-treated 1X TBE, run at 270 volts for 10 minutes) with 1 ug and 0.5 ug of RNA standard (Stratagene catalogue #735026: Adult, Total Placenta RNA ) . The RNA samples are quantified by comparing intensity of sample bands to intensity of standard bands using a densitometer, Alpha Imager 2200.

Probes derived from the collected RNA can be labeled using standard methods (e.g., reverse transcription and PCR in the presence of labeled nucleotides).

A variety of well known methods, e.g., amplification and hybridization-based methods, are suitable for detecting IRM gene expression (see, e.g., Sambrook and Ausubel, supra), and any method suitable to the sample may be used. For example, hybridization based assays (assays in which a polynucleotide probe is hybridized to a target polynucleotide) may be used. Exemplary polynucleotide probes and primers are described infra, and methods of selecting polynucleotide probe sequences for use in polynucleotide hybridization are well known (see, e.g., Sambrook and Ausubel, supra).

In some hybridization formats, at least one of the target and probe is immobilized. The immobilized polynucleotide may be DNA, RNA, or another oligo- or poly-nucleotide, and may comprise natural or non-naturally occurring nucleotides, nucleotide analogs, or backbones. Such assays may be in any of several formats including high-density polynucleotide or oligonucleotide arrays (Lipshutz, et. al. Nat Genet 1999, 21:20-4; U.S. Pat. Nos. 5,445,934; 5,578,832; 5,556,752; and 5,510,270), high density cDNA arrays (see, e.g., Schena et al., 1995, Science 270:467-70), Southern, Northern, dot and slot blots, dip sticks, pins, chips, or beads. All of these techniques are well known in the art and are the basis of many commercially available diagnostic kits.

Hybridization techniques are generally described in Hames et al., ed., Nucleic Acid Hybridization, A Practical Approach IRL Press, (1985); Gall and Pardue, 1969, Proc. Natl. Acad. Sci., U.S.A., 63:378-383; and John et al., 1969, Nature, 223:582-587.

Dot blots may be used to determine the amount of IRM transcript present in a nucleic acid sample obtained from an individual being tested. In these assays, a sample from an individual being tested is spotted on a support (e.g., a filter) and then probed with labeled nucleic acid probes that specifically hybridize with IRM nucleic acids. After the probes have been allowed to hybridize to the immobilized nucleic acids on the filter, unbound nucleic acids are rinsed away and the presence of hybridization complexes detected and quantitated on the basis of the amount of labeled probe bound to the filter.

Northern blots can be used to detect and quantitate a IRM transcript in a sample. Such methods typically involve initially isolating total cellular or poly(A) RNA and separating the RNA on an agarose gel by electrophoresis. The gel is then overlaid with a sheet of nitrocellulose, activated cellulose, or glass or nylon membranes and the separated RNA transferred to the sheet or membrane by passing buffer through the gel and onto the sheet or membrane. The presence and amount of IRM transcript present on the sheet or membrane can then be determined by probing with a labeled probe complementary to IRM to form labeled hybridization complexes that can be detected and optionally quantitated (see, e.g., Sambrook and Ausubel, supra).

Related hybridization methods utilize nucleic acid probe arrays to detect and quantitate IRM transcripts. The probes utilized in the arrays can be of varying types and can include, for example, synthesized probes of relatively short length (e.g., a 20-mer or a 25-mer), cDNA (full length or less-than-full length fragments of gene) , amplified DNA, fragments of DNA (generated by restriction enzymes, for example) and reverse-transcribed DNA (see, e.g., Southern et al., 1999, Nature Genetics Supplement 21:5-9). Both custom and generic arrays can be utilized in detecting IRM expression levels. Custom arrays can be prepared using probes that hybridize to particular preselected subsequences of mRNA gene sequences of IRM or amplification products prepared from them. Generic arrays are not specially prepared to bind to IRM sequences but instead are designed to analyze mRNAs irrespective of sequence. Nonetheless, such arrays can still be utilized because IRM nucleic acids only hybridize to those locations that include complementary probes. Thus, IRM levels can still be determined based upon the extent of binding at those locations bearing probes of complementary sequence.

In probe array methods, once nucleic acids have been obtained from a test sample, they typically are reversed transcribed into labeled cDNA, although labeled mRNA can be used. The test sample containing the labeled nucleic acids is then contacted with the probes of the array. After allowing a period sufficient for any labeled IRM nucleic acid present in the sample to hybridize to the probes, the array is typically subjected to one or more high stringency washes to remove unbound nucleic acids and to minimize nonspecific binding to the nucleic acid probes of the arrays. Binding of labeled IRM is detected using any of a variety of commercially available scanners and accompanying software programs.

For example, if the nucleic acids from the sample are labeled with fluorescent labels, hybridization intensity can be determined by, for example, a scanning confocal microscope in photon counting mode. Appropriate scanning devices are described by e.g., U.S. 5,578,832 to Trulson et al., and U.S. 5,631,734 to Stem et al. and are available from Affymetrix, Inc., under the GeneChip™ label. Some types of label provide a signal that can be amplified by enzymatic methods (see Broude, et al., 1994, Proc. Natl. Acad. Sci. U.S.A. 91:3072-76). A variety of other labels are also suitable including, for example, radioisotopes, chromophores, magnetic particles and electron dense particles.

Those locations on the probe array that are hybridized to labeled nucleic acid are detected using a reader, such as described by U.S. Patent No. 5,143,854, WO 90/15070, and U.S. 5,578,832. For customized arrays, the hybridization pattern can then be analyzed to determine the presence and/or relative amounts or absolute amounts of known mRNA species in samples being analyzed as described in e.g., WO 97/10365. Further guidance regarding the use of probe arrays sufficient to guide one of skill in the art is provided in WO 97/10365, PCVUS/96/143839 and WO 97/27317. Additional discussion regarding the use of microarrays in expression analysis can be found, for example, in Duggan, et al., 1999, Nature Genetics Supplement 21:10-14; Bowtell, 1999, Nature Genetics Supplement 21:25-32; Brown and Botstein, 1999, Nature Genetics Supplement 21:33-37; Cole et al., 1999, Nature Genetics Supplement 21:38-41; Debouck and Goodfellow, 1999, Nature Genetics Supplement 21:48-50; Bassett, Jr., et al., 1999, Nature Genetics Supplement 21:51-55; and Chakravarti, 1999, Nature Genetics Supplement 21:56-60.

Ribonuclease protection assays (RPA) can be used to detect IRM expression. RPA involve preparing a labeled antisense RNA probe for IRM. This probe is subsequently allowed to hybridize in solution with IRM transcript contained in a biological sample to form RNA:RNA hybrids. Unhybridized RNA is then removed by digestion with an RNAase, while the RNA:RNA hybrid is protected from degradation. The labeled RNA:RNA hybrid is separated by gel electrophoresis and the band corresponding to IRM detected and quantitated. Usually the labeled RNA probe is radiolabeled and the IRM band detected and quantitated by autoradiography. RPA is discussed further by (Lynn et al., 1983, Proc. Natl. Acad. Sci. 80:2656; Zinn et al., 1983, Cell 34:865; and Sambrook and Ausubel, supra).

In one embodiment, in situ hybridization is used to detect IRM sequences in a sample. In situ hybridization assays are well known and are generally described in Angerer et al., Methods Enzymol., 152: 649-660 (1987) and Ausubel, supra. The method usually involves initially fixing test cells to a support (e.g., the walls of a microtiter well) and then permeabilizing the cells with an appropriate permeabilizing, solution. A solution containing labeled probes for IRM is then contacted with the cells and the probes allowed to hybridize with IRM nucleic acids. Excess probe is digested, washed away and the amount of hybridized probe measured. This approach is described in greater detail by Harris, 1996, Anal. Biochem. 243:249-256; Singer et al., 1986, Biotechniques 4:230-250; Haase et al., 1984, Methods In Virology, vol. VII, pp. 189-226; and Nucleic Acid Hybridization: A Practical Approach (Hames, et al., eds., 1987).

Amplification-based methods such as PCR and LCR are also useful for detection of IRM expression. A variety of methods are known for amplifying nucleic acids, for example, (1) the polymerase chain reaction (PCR) [see, e.g., PCR Technology: Principles and Applications for DNA Amplification (H.A. Erlich, Ed.) Freeman Press, NY, NY (1992); PCR Protocols: A Guide to Methods and Applications (Innis, et al., Eds.) Academic Press, San Diego, CA (1990); and U.S. Patent Nos. 4,683,202 and 4,683,195]; (2) the ligase chain reaction (LCR) [see, e.g., Wu and Wallace, Genomics 4:560 (1989) and Landegren et al., Science 241:1077 (1988)]; (3) transcription amplification (see, e.g., Kwoh et al., Proc. Natl. Acad. Sci. USA 86:1173 (1989)]; (4) self-sustained sequence replication [see, e.g., Guatelli et al., Proc. Natl. Acad. Sci. USA, 87:1874 (1990)]; and (5) nucleic acid based sequence amplification (NABSA) [see, e.g., Sooknanan, R. and Malek, L., BioTechnology 13:563-65 (1995)]; (6) strand displacement amplification (SDA; e.g., Walker et al., 1992, Proc. Natl. Acad. Sci. U.S.A. 89:392-396); (7) the nucleic acid sequence based amplification (NASBA, Cangene, Mississauga, Ontario; e.g., Compton, 1991, Nature 350:91), and the like.

One useful variant of PCR is PCR ELISA (e.g., Boehringer Mannheim Cat. No. 1 636 111) in which digoxigenin-dUTP is incorporated into the PCR product. The PCR reaction mixture is denatured and hybridized with a biotin-labeled oligonucleotide designed to anneal to an internal sequence of the PCR product. The hybridization products are immobilized on streptavidin coated plates and detected using anti-digoxigenin antibodies.

A variety of so-called "real time amplification" methods or "real time quantitative PCR"methods can also be utilized to determine the quantity of IRM mRNA present in a sample. Such methods involve measuring the amount of amplification product formed during an amplification process. Fluorogenic nuclease assays are one specific example of a real time quantitation method that can be used to detect and quantitate IRM transcripts. In general such assays continuously measure PCR product accumulation using a dual-labeled fluorogenic oligonucleotide probe, an approach frequently referred to in the literature simply as the "TaqMan"method. The probe used in such assays is typically a short (ca. 20-25 bases) polynucleotide that is labeled with two different fluorescent dyes. The 5" terminus of the probe is typically attached to a reporter dye and the 3" terminus is attached to a quenching dye, although the dyes can be attached at other locations on the probe as well. For measuring an IRM transcript, the probe is designed to have at least substantial sequence complementarity with a probe binding site on an IRM transcript. Upstream and downstream PCR primers that bind to regions that flank IRM are also added to the reaction mixture for use in amplifying the IRM polynucleotide. When the probe is intact, energy transfer between the two fluorophors occurs and the quencher quenches emission from the reporter. During the extension phase of PCP, the probe is cleaved by the 5" nuclease activity of a nucleic acid polymerase such as Taq polymerase, thereby releasing the reporter dye from the polynucleotide-quencher complex and resulting in an increase of reporter emission intensity that can be measured by an appropriate detection system.

Primers useful for amplification-based detection can be readily designed based on knowledge of the target sequence (sequence to be detected). Particularly suitable primers for some assays have a T_{M} close to 60^{o}C, are between 100 and 600 bp in length and are specific for the region to be amplified (which can be determined by BLAST analysis of GenBank and the prospective primers, for example using software such as Oligo 6 (Molecular Biology Insights, Inc.; http://www.oligo.net). Preferably primers span an intron/exon splice junction so that amplification of desired RNA/cDNA can be easily separated from that of contaminating genomic DNA. It is well known that primers should be selected that do not form duplexes within themselves or with the other primer of the pair (if present) used for amplification.

One detector which is specifically adapted for measuring fluorescence emissions such as those created during a fluorogenic assay is the ABI 7700 manufactured by Applied Biosystems, Inc., in Foster City, CA. Computer software provided with the instrument is capable of recording the fluorescence intensity of reporter and quencher over the course of the amplification. These recorded values can then be used to calculate the increase in normalized reporter emission intensity on a continuous basis and ultimately quantify the amount of the mRNA being amplified.

In another example of a real-time PCR method, PCR is carried out with a Cy5 labeled primer and a single fluorescein-labeled probe. When the probe is annealed to the extension product of the Cy5-labeled primer, the flourophores are brought into close enough contact for resonance energy transfer to occur, increasing the fluorescence of the Cy5. See, Stoitchkov et al., Clin Chim Acta. 2001 306: 133-8.

Another detection method that can be used with multiple instrument systems makes use of molecular beacons. Molecular beacons are DNA molecules with an internally quenched fluorophore whose fluorescence is restored when they bind to a complimentary target. Molecular beacons consist of a loop and stem structures. The loop portion of the molecule is a probe sequence complementary to a target DNA sequence. The stem is formed by the annealing of complementary sequences on the ends of the probe sequence. A fluorescent molecule is attached to one end of the DNA sequence and a quenching molecule is attached to the opposite end. The hybridization of the stem keeps these two molecules in close proximity to each other, causing the fluorescence of the fluorophore to be quenched by resonance energy transfer. When the probe encounters a target molecule, it hybridizes to the complementary sequence. This hybridization forces the stem apart and causes the fluorophore and the quencher to move away from each other, leading to the restoration of fluorescence that can be detected. See, Steuerwald et al., 1999, Mol Hum Reprod 5:1034-39.

Additional details regarding the theory and operation of fluorogenic methods for making real time determinations of the concentration of amplification products are described, for example, in U.S. Pat Nos. 5,210,015 to Gelfand, 5,538,848 to Livak, et al., and 5,863,736 to Haaland, as well as Heid et al., 1996, Genome Research, 6:986-994; Gibson et al., 1996, Genome Research 6:995-1001; Holland et al., 1991, Proc. Natl. Acad. Sci. USA 88:7276-7280; and Livak et al., 1995, PCR Methods And Applications 357-362.

As noted supra, it is sometimes desirable to establish a standard reference cDNA to which expression of IRM gene product in a subject is compared. Suitable standard reference cDNA can be prepared in a variety of ways that will be apparent to the skilled practitioner. For illustration, one such standard may be a pre-made cDNA sample derived from RNA of a pool of IS subjects who have similar OGTT and SSPG values as the extreme IS phenotype population. Fasting blood samples are collected from each of these eIS control subjects, and total RNA extracted within one-hour of blood collection using standard methods (e.g. Trizol method by Gibco-BRL). Equal amounts of RNA from each IS standard subject is pooled and labeled with either Cy3-deoxyuridine triphosphate (dUTP) for initial test or Cy5-dUTP for a confirmation test. To evaluate the gene expression profile in a patient subject, fasting blood is collected and RNA extracted under identical conditions. The RNA is used to make cDNA labeled with Cy5-dUTP. The cDNA is mixed with equal amount of Cy3-labeled standard cDNA, and hybridized to a microarray (glass or membrane) that contains a probe(s) for one or more IRM genes. The level of gene expression relative to the standard control is determined using methods described above and the patients risk for developing IR or IR related conditions may be scored based on the combined number of genes that are either up or down regulated as compared to the standard control. If desired, the result may be confirmed with the "flip-dye" technique as described in the Examples (see, e.g. Wang et al., 2000, Nat Biotech.18:457-59).

It will be apparent that, in alternative embodiments the standard pre-made cDNA sample can be made from healthy ("normal") subjects, insulin resistant subjects, insulin sensitive subjects, and the like. In general, similarity of the (relative) expression level of an IRM gene a patient and standard is indicative that the patient has the same phenotype (e.g., normal, insulin resistant) as the standard.

Nucleic Acid Primers and Probes

The primers and hybridization probes utilized in the foregoing methods are polynucleotides that are of sufficient length to specifically hybridize (e.g. under stringent conditions) an IRM gene mRNA transcript in the sample. As noted above, one of skill will be able to select and prepare suitable probes or primers for detection of the IRM mRNA. In an embodiment, for example, a primer or probe may hybridize to, for example, (1) a polynucleotide having an accession sequence of Table 1 or its complement (excluding any poly(A) tail) as well as to (2) a polynucleotide having the sequence of the insert of an IMAGE clone listed in Table 1. In various embodiments, the probes have substantial sequence identity to a polynucleotide of (1) or (2) described above it the complement thereof. In various embodiments, probes hybridize under stringent conditions to a complement of a polynucleotide sequence of (1) or (2) described above. In various embodiments, probes comprise at least 10 bases identical to or exactly complementary to a polynucleotide of (1) or (2) described above, often at least about 15 bases, at least about 20 bases, at least about 25 bases, at least about 50 bases, at least about 100, or at least about 500 bases. Primers often contain between about 12 and about 100 contiguous nucleotides identical or exactly complementary to an IRM sequence, more often between about 12 and about 50 contiguous nucleotides, even more often between about 15 and about 25 contiguous nucleotides. Probes can be designed based on the sequence of a naturally occurring mRNA that comprises a polynucleotide referred to in Table 1 or a fragment thereof.

Hybridization probes are typically at least 15 nucleotides in length, in some instances 20 to 30 nucleotides in length, in other instances 30 to 50 nucleotides in length, and in still other instances up to the full length of a IRM nucleic acid. In some embodiments, primers and hybridization probes are less than about any of the following lengths (in bases or base pairs): 10,000; 5,000; 2500; 2000; 1500; 1250; 1000; 750; 500; 300; 250; 200; 175; 150; 125; 100; 75; 50; 25; 10. In some embodiments, a primer or hybridization probe is greater than about any of the following lengths (in bases or base pairs): 10; 15; 20; 25; 30; 40; 50; 60; 75; 100; 125; 150; 175; 200; 250; 300; 350; 400; 500; 750; 1000; 2000; 5000; 7500; 10000; 20000; 50000. Alternately, a primer or hybridization probe can be any of a range of sizes having an upper limit of 10,000; 5,000; 2500; 2000; 1500; 1250; 1000; 750; 500; 300; 250; 200; 175; 150; 125; 100; 75; 50; 25; or 10 and an independently selected lower limit of 10; 15; 20; 25; 30; 40; 50; 60; 75; 100; 125; 150; 175; 200; 250; 300; 350; 400; 500; 750; 1000; 2000; 5000; 7500 wherein the lower limit is less than the upper limit. In various embodiments, a probe sequence, or a portion delineated above, has a sequence identical or exactly complementary to an IRM sequence.

In some embodiments, the probes and primers are modified, e.g., by adding restriction sites to the probes or primers. In other embodiments, primers or probes of the invention comprise additional sequences, such as linkers. In still some other embodiments, primers or probes of the invention are modified with detectable labels. For example, the primers and probes are chemically modified, e.g., derivatized, incorporating modified nucleotide bases, or containing a ligand capable of being bound by an anti-ligand (e.g., biotin). In some embodiments, the probes are labeled with a detectable label, such as a radiolabel, fluorophore, chromophore or enzyme to facilitate detection. In some embodiments, the probes are derivitized. The primers and probes of the invention may be prepared by routine methods including chemical synthesis (see, e.g., Narang et al., 1979, Methods of Enzymology 68:90; Brown et al., 1979, Methods of Enzymology 68:109) or recombinant methods. Primers and probes may be RNA, DNA, PNA or chimeric, and may contain non-naturally occurring bases, e.g., deoxyinosine (see, Batzer et al., 199 1, Nucleic Acid Res. 19:5081; Ohtsuka et al., 1985, J Biol. Chem. 260:2605-2608; Rossolini et al., 1994, Mol. Cell. Probes 8:91-98) or modified backbone residues or linkages. Provided with the guidance herein, one of skill will be able to select primer pairs that specifically amplify all or a portion of an IRM gene, mRNA, or cDNA in a sample.

Assays for IRM Polypeptides Expression of IRM polypeptides can also be detected. As used herein, the term IRM polypeptide refers to a polypeptide (e.g., a naturally occurring polypeptide) encoded by an IRM gene described herein. The term IRM polypeptide also includes allelic variants and modified proteins. In some embodiments, the term IRM also includes truncated or variant polypeptides encoded by partial sequences (e.g., an expressed sequence tag). Exemplary polypeptide sequences will be apparent by reference to the annotations accompanying the GenBank accession numbered sequences provided in Table 1, and can be deduced by conceptual translation of the polynucleotide sequences disclosed herein. IRM proteins can be isolated from tissues (e.g., blood) using protein isolation well known to those of skill (e.g., such as those described in Harlow and Lane, supra. Methods for detecting a specified polypeptide are well known and include, without limitation, enzyme immunoassay (EIA), radioimmunoassay (RIA), Western blot analysis, immunohistochemistry and enzyme linked immunoabsorbant assay (ELISA). It will be appreciated that it is not always necessary to isolate the IRM proteins; for example, often the proteins are assayed in a cell lysate or even as expressed on the surface of the cells of the tissue. Guided by the disclosure herein of the correlation between IRM expression and insulin resistance and related conditions, the ordinarily skilled practitioner can design assays to detect (qualitatively or quantitatively) IRM polypeptide expression.

In one embodiment, immunological methods are used, for example using an antibody or other specific binding agent that binds the IRM polypeptide. Anti-IRM antibodies (monoclonal or polyclonal) can be made by a variety of means well known to those of skill in the art. See, e.g., Harlow and Lane, supra, Coligan et al., supra. These techniques include antibody preparation by selection of antibodies from libraries of recombinant antibodies in phage or similar vectors. See, Huse et al., 1989, Science 246:1275-81; and Ward et al., 1989, Nature 341:544-46. To produce anti-IRM antibodies, an IRM polypeptide or, more often, an immunogenic fragment thereof, is used as an immunogen or for screening of IRM binding fragments. IRM polypeptides or fragments can be prepared by recombinant expression or chemical synthesis, as described elsewhere herein. For production of polyclonal antibodies, an appropriate target immune system is selected, typically a mouse or rabbit, but also including goats, sheep, cows, chickens, guinea pigs, monkeys and rats. The immunoglobulins produced by the host can be precipitated, isolated and purified by routine methods, including affinity purification. Substantially monospecific antibody populations can be produced by chromatographic purification of polyclonal sera.

A number of well-established immunological binding assays are suitable for detecting and quantifying IRM of the present invention. See, e.g., U.S. Patents 4,366,241; 4,376,110; 4,517,288; and 4,837,168, and also Methods In Cell Biology Volume 37: Antibodies In Cell Biology, Asai, ed. Academic Press, Inc. New York (1993); Basic and Clinical Immunology 7th Edition, Stites & Terr, eds. (1991); Harlow and Lane, supra, Coligan, and Ausubel, supra.

Immunoassays for detecting IRM polypeptides may be competitive or noncompetitive. Usually the IRM gene product being assayed is detected directly or indirectly using a detectable label. The particular label or detectable group used in the assay is usually not a critical aspect of the invention, so long as it does not significantly interfere with the specific binding of the antibody or antibodies used in the assay. The label may be covalently attached to the capture agent (e.g., an anti-IRM antibody), or may be attached to a third moiety, such as another antibody, that specifically binds to the IRM polypeptide at a different epitope than recognized by the capture agent.

Noncompetitive immunoassays are assays in which the amount of captured analyte (here, the IRM polypeptide) is directly measured. One such assay is a two-site, monoclonal-based immunoassay utilizing monoclonal antibodies reactive to two non-interfering epitopes on the captured analyte. See, e.g., Maddox et al., 1983, J Exp. Med., 158:1211 for background information. In such an assay, the amount of IRM in the sample is directly measured. For example, using a so-called "sandwich" assay, the capture agent (here, the anti-IRM antibodies) can be bound directly to a solid substrate where they are immobilized. These immobilized antibodies then capture polypeptide present in the test sample. IRM thus immobilized is then bound by a labeling agent, such as a second IRM antibody bearing a label. Alternatively, the second IRM antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin. Certain of the sandwich assays are enzyme-linked immunosorbent assays (ELISA) in which the detection antibody bears an enzyme. The detection antibody is detected by providing a substrate for the enzyme to generate a detectable signal.

In competitive assays, the amount of IRM polypeptide present in the sample is measured indirectly by measuring the amount of an added (exogenous) IRM polypeptide displaced (or competed away) from a capture agent (e.g., anti-IRM antibody) by the analyte present in the sample (e.g., IRM polypeptide). In one competitive assay, a known amount of IRM is added to the sample and the sample is then contacted with a capture agent (e.g., an anti-IRM antibody) that specifically binds to IRM. The amount of IRM bound to the antibody is inversely proportional to the concentration of IRM present in the sample.

Preferably, the antibody is immobilized on a solid substrate. The amount of IRM bound to the antibody may be determined either by measuring the amount of IRM present in an IRM/antibody complex, or alternatively by measuring the amount of remaining uncomplexed IRM. The amount of IRM may be detected by providing a labeled IRM molecule.

For example, using the hapten inhibition assay, the analyte (in this case IRM) is immobilized on a solid substrate. A known amount of anti-IRM antibody is added to the sample, and the sample is then contacted with the immobilized IRM. In this case, the amount of anti-IRM antibody bound to the immobilized IRM is inversely proportional to the amount of IRM present in the sample. Again the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Further guidance regarding the methodology and steps of a variety of antibody assays is provided, for example, in U.S. Patent No. 4,376,110 to Greene; "Immunometric Assays Using Monoclonal Antibodies,"in Antibodies: A Laboratory Manual, Cold Spring Harbor Laboratory, Chap. 14 (1988); Bolton and Hunter, "Radioimmunoassay and Related Methods,"in Handbook of Experimental Immunology (D.M. Weir, ed.), Vol. 1, chap. 26, Blackwell Scientific Publications, 1986; Nakamura, et al., "Enzyme Immunoassays: Heterogeneous and Homogenous Systems,"in Handbook of Experimental Immunology (D.M. Weir, ed.), Vol. 1, chap. 27, Blackwell Scientific Publications, 1986; Coligan, supra.The antibodies used to perform the foregoing assays can include polyclonal antibodies, monoclonal antibodies and fragments thereof as described infra. Monoclonal antibodies can be prepared according to established methods (see, e.g., Kohler and Milstein (1975) Nature 256:495; and Harlow and Lane, supra.In addition to the competitive and non-competitive IRM polypeptide immunoassays, the present invention also provides other assays for detection and quantification of IRM polypeptides. For example, Western blot (immunoblot) analysis can be used to detect and quantify the presence of IRM in the sample. The technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the sample with the antibodies that specifically bind IRM. The anti-IRM antibodies specifically bind to IRM on the solid support. These antibodies may be directly labeled or alternatively may be subsequently detected using labeled antibodies (e.g., labeled sheep anti-mouse antibodies) that specifically bind to the anti-IRM.

Furthermore, assays such as liposome immunoassays (LIA) are also encompassed by the present invention. LIA utilizes liposomes that are designed to bind specific molecules (e.g., antibodies) and to release encapsulated reagents or markers. The released chemicals are then detected according to standard techniques (see, Monroe et al., 1986, Amer. Clin. Prod. Rev. 5:34-41).

Various IRM activities can also be determined to detect a change in increase in IRM polypeptide expression. For example, when the IRM has an assayable enzymatic activity, an increase in enzyme activity is indicative of increased IRM expression. In one assay, a metabolite which is produced directly (i.e., catalyzed) or indirectly by an IRM protein is detected.

Time Course AnalysesCertain prognostic methods of assessing a patient"s risk of insulin resistance and related conditions involve monitoring IRM expression levels for a patient susceptible to insulin resistance or IR-related conditions to track whether there appears to be a change in IRM expression over time. An change in IRM expression over time can indicate that the individual is at increased risk for developing insulin resistance or related conditions. As with other measures of IRM, the IRM expression level for the patient at risk for IRM can compared against a reference (or baseline) value. The baseline in such analysis can be a prior value determined for the same individual or a statistical value (e.g., mean or average) determined for a control group (e.g., a population of individuals with no apparent risk factors, a eIS phenotype population, etc.). An individual showing a statistically significant increase in IRM expression levels over time can prompt the individual"s physician to take prophylactic measures to lessen the individual"s potential for developing insulin resistance.

Evaluation of Therapeutic TreatmentThe assays of the invention may also be used to evaluate the efficacy of a particular therapeutic treatment regime in animal studies, in clinical trials, or in monitoring the treatment of an individual patient. In these cases, it may be desirable to establish the baseline for the patient prior to commencing therapy and to repeat the assays one or more times through the course of treatment, usually on a regular basis, to evaluate whether IRM levels are moving toward the desired endpoint as a result of the treatment. Thus, the invention provides a method of assessing the efficacy of a therapy for reducing or treating insulin resistance in a patient by comparing expression of an IRM gene product in a first sample obtained from the patient prior to providing at least a portion of the therapy to the patient expression of the marker in a second sample obtained from the patient following provision of the portion of the therapy, wherein a statistically significant change in expression of the IRM gene (or preferably, at least 2, at least 3, or at least 4 IRM genes) is an indication that the therapy is efficacious for treatment of insulin resistance.

The assays of the invention are also useful for conducting clinical trials of drug candidates for insulin resistance and associated metabolic diseases. Such trials are performed on treated or control populations having similar or identical expression profiles at a defined collection of genes. Use of genetically matched populations eliminates or reduces variation in treatment outcome due to genetic factors, leading to a more accurate assessment of the efficacy of a potential drug.

Furthermore, the assays of the invention may be used after the completion of a clinical trial to elucidate differences in response to a given treatment. For example, one or more of the IRM genes and/or associated polymorphisms may be used to stratify the enrolled patients into disease sub-types or classes. It may further be possible to use the genes to identify subsets of patients with similar expression profiles who have unusual (high or low) response to treatment or who do not respond at all (non-responders). In this way, information about the underlying genetic factors influencing response to treatment can be used in many aspects of the development of treatment (these range from the identification of new targets, through the design of new trials to product labeling and patient targeting). Additionally, the IRM genes may be used to identify the genetic factors involved in an adverse response to treatment (adverse events). For example, patients who show adverse responses may have more similar expression profiles than would be expected by chance. This would allow the early identification and exclusion of such individuals from treatment. It would also provide information that might be used to understand the biological causes of adverse events and to modify the treatment to avoid such outcomes.

Detection of Polymorphisms Associated With Susceptibility to Insulin Resistance Based on the teachings of the present invention, polymorphisms in one or more of the IRM genes listed in Tablethat correlate with insulin resistance or related phenotypes in a population can be identified. Polymorphism refers to the occurrence of two or more genetically determined alternative sequences (called alleles) for a specific gene in a population. Some polymorphisms in IRM genes are expected to be associated with the several biological and medical conditions associated with insulin resistance including diabetes and syndrome X. Such polymorphisms can be used for a number of prognostic and diagnostic methods.

In one embodiment, polymorphisms useful in screening are identified by comparing the sequence (e.g., a cDNA sequence, a genomic sequence including promoter sequence and introns, or portions of either) of IRM genes from populations of subjects who differ in insulin resistance phenotype. As used herein, the term "phenotype" refers to any detectable or otherwise measurable property of an organism (e.g., patient) such as symptoms of, or susceptibility to a disease such as insulin resistance or an insulin resistance related condition (e.g., syndrome X or diabetes). Examples of populations of subjects who differ in insulin resistance phenotype include, but are not limited to (1) eIS phenotype subjects and eIR phenotype subjects, (2) subjects who are or are not insulin resistant, (3) subjects who are and are not deemed at increased at risk for developing insulin resistance (4) subjects who suffer from and subjects who do not suffer from a insulin resistance related condition such as diabetes, (5) subjects who are at increased risk for and subjects not at increased risk for developing insulin resistance, or (6) combinations of populations in different groups listed. For purposes of clarity and not limitation, the exemplary populations with eIS and eIR phenotypes will be referred to below. However, each such reference should be understood to refer to other IR-related phenotypes as well.

Polymorphic markers include restriction fragment length polymorphisms (RFLPs), variable number of tandem repeats (VNTRs), hypervariable regions, microsatellites, simple sequence repeats (di-, tri-, or tetra-nucleotide). A single nucleotide polymorphism (SNP) occurs at a polymorphic site occupied by a single nucleotide, which is the site of variation between allelic sequences. The site is usually flanked by highly conserved sequences of the gene. The allelic form occurring most frequently in a selected population is sometimes referred to as the wildtype form. Diploid organisms, such as humans, may be homozygous or heterozygous for allelic forms.

Polymorphic forms of one or more genes listed in Tableare expected to correlate with insulin resistance and will be useful in identifying individuals at risk for these disorders. Preferred polymorphic markers have at least two alleles, each occurring at frequency of greater than 1%, and more preferably greater than 10% of a selected population. The determination of a sequence or of polymorphisms in IRM genes of an individual or population is sometimes referred to herein as "genotyping." Genotyping comprise determining the identity of a polymorphism in an IRM gene by any method known in the art. Polymorphisms can be identified by direct sequencing. For assays of genomic DNA, virtually any biological sample containing DNA is suitable. For assays of cDNA, a tissue sample will be obtained from an organ in which the IRM are expressed (e.g. the white blood cells). Purified genomic DNA or cDNA are amplified by PCR using a set of overlapping primers specifically designed to amplify the genomic DNA or cDNA in a series of overlapping fragments of 500-1000 bp spanning the entire gene (including promoter sequence) or cDNA. Putative polymorphisms within these amplified PCR fragments between eIS and eIR individuals can be detected using any of a variety of standard methods, e.g., (1) Direct-sequencing analysis using either the dideoxy-chain termination method or the Maxam-Gilbert method (see, e.g., Sambrook, Ausubel, supra), (2) SSCP (Orita et al. PNAS 86:2766-2770 (1989), (3) Denaturing Gradient Gel Electrophoresis (PCR Technology, Principles and Application for DNA amplification, Chapter 7, Henry Erlich, ed. W.H.Freeman and Co. New York, 1992, and other methods well known in the art (e.g., single strand polymorphism assay, ligase chain reaction, enzymatic cleavage, and Southern hybridization).

Alternatively, or in conjunction with DNA sequencing, other methods are useful for identification of changes in IRM genes. Methods include: single strand polymorphism assay ("SSPA") analysis and heteroduplex analysis methods (Orita et al., 1989, Proc Natl Acad Sci USA, 86:2766); ligase chain reaction (LCR); mismatch detection protocols; testing for the presence or form of the protein produced by the gene (e.g., by isoelectric focusing and/or immunoassay). The polymorphism in the IRM gene may be a single base substitution resulting in an amino acid substitution or a translational stop, an insertion, a deletion, or a gene rearrangement. The polymorphism may be located in an intron, an exon of the gene, or a promoter or other regulatory region which affects the expression of the gene. Examples of polymorphisms identified by sequencing IRM 10 (hypothetical protein FLJ22297) are described in Table 2 (additional data concerning the SNP at +686 was also found at the National Center for Biotechnology Information (NCBI) database.

| | | | |
|---|---|---|---|
| | SNP and flanking sequence | Forward PCR Primer Reverse PCR Primer | Allele frequency |
| a) SNP location: 5' UTRb) SNP alleles and nucleotide location: C (-187) Tc) PCR product size(bp): 138 | AAAGAAAACTGCTGCAGATGGAAAAAGGCAAGAGATCATTGTTCTGGATTCCAAGAGGAGTAA(C/T)GCCATCAATATTGGTCTGACGGTGCTGCCCCCTCCAAGGACGATTAAGATCGCC | F: GAA AAA GGC AAG AGA TCA TT R: TTC CTT CTT TGT TTA AGG CA | C: 69%T: 31%16 Chrom. |
| a) SNP Location: CDSb) SNP alleles and nucleotide location:C (+686) Tc) PCR product size(bp): 146d) NCBI SNP numberrs2303510 | CTGACCTGGT GATGGCCCCGATCTCCGAGTACAGATCGGAGCTGTCTGGGAAGTTTTCTA(G/A)CACCATGGTGCACACATGGTGGAGAAGCGACTGCTTGTGCACTGTGTCTTTGACTTCTGG | F: GCC AAA GCG TTT GAG TTA AG R: ATG GCC CCG ATC TCC GAG TA | T: 30%C:70%1496 Chrom. |

In another embodiment, polymorphisms useful in screening are identified by reviewing polymorphisms described in public databases as being present in the IRM-genes disclosed herein. Further, putative polymorphisms identified by database searches of IRM genes (e.g., a search of the SNP consortium database; www.ncbi.nlm.nih.gov/SNP) or by other methods may be verified by DNA sequencing to determine the exact nature of the polymorphisms. Examples of polymorphisms in coding regions of selected IRM genes identified from public databases are described in Table 3.

### Table 3

After determining polymorphisms present in these groups of individuals at one or more polymorphic sites in one or more IRM genes, the information is analyzed to detect correlation between specific allele(s) of one of more IRM genes and an insulin resistance phenotypes. In one embodiment, this analysis is carried out by determining the frequency of each polymorphic allele in one or more IRM genes are compared between the eIS and eIR individuals and the polymorphisms with different allele frequency between the two groups will be selected for further testing in a large group of individuals (n=250-500). The standard chi-square test can be used to identify statistically significant correlation (p<0.05) between one or more of these alleles and insulin resistance (e.g., as determined by standard assays). For illustration, it might be found that the frequency of A1 allele at polymorphic site A of gene X of the IRM gene is higher in individuals in the eIR group as compared to those in the eIS group in the initial screen. This difference in A1 allele frequency is found to be statistically significant and correlates with insulin resistance in the large set of 250-500 individuals. Furthermore, it might be found that the combined presence of allele A1 at polymorphic site A of gene X and allele B1 at polymorphic site B of gene Y correlate more significantly with an insulin resistance phenotype in this group of individuals as judged by a more significant P values (e.g. P of 0.05 in the single polymorphism test vs P of 0.005 in the double polymorphism test).

Methods for conducting association studies, haplotype determination method, are known and are described in, for example WO 01/64957 (Polymorphisms Associated with insulin-Signaling and Glucose-Transport Pathways) and U.S. patent no. 6,346,381, both of which are incorporated herein by reference.

Thus, in one embodiment, the invention provides a method for assessing a subject"s risk of developing insulin resistance by detecting at least one polymorphism in an IRM gene in the individual that is correlated with a IRM polymorphism associated with insulin resistance.

Combined detection of several such polymorphic forms from one or more genes listed in Tablewill increase the confidence in the diagnosis. For example, the presence of a single IRM polymorphic form known to correlate with insulin resistance might indicate a (hypothetical) probability of 20% that an individual has or is susceptible to developing insulin resistance, whereas detection of multiple (e.g., five) polymorphic forms, each of which correlates with a 20% probability of susceptibility, will usually indicate a much higher probability (e.g., 80%) that the individual has or is susceptible to insulin resistance or related conditions. A combination of alleles present in an individual or a sample is referred to as a "haplotype." In the context of the present invention a haplotype refers to a combination of more than one IRM gene associated polymorphisms (alleles) found in a given individual and which is associated with a phenotype (e.g., greater than average susceptibility to insulin resistance). Analysis of the IRM polymorphisms can be combined with analysis of other polymorphisms or other risk factors of insulin resistance, such as personal and/or family history of type II diabetes, etc.

In some embodiments, the assay comprises detecting the presence (or absence) of polymorphism markers for two or more IRM genes (e.g., a panel of at least 4, 5, 6, 7, 8, 9, 10, 11, 12, 15, 20, or 25).

Thus, in one aspect, the invention provides a method of determining whether an individual is at risk of developing insulin resistance or whether said individual suffers from insulin resistance by obtaining a nucleic acid sample from the individual and determining whether the nucleotides present at one or more IRM genes are indicative of a risk of developing insulin resistance.

In one aspect, the invention provides a method of estimating the frequency of an allele in a population of eIR or eIS individuals by obtaining a nucleic acid sample from each of a plurality of individuals in said population, and determining the proportional representation of a polymorphic base in an IRM gene in the pooled nucleic acid sample derived from said population.

In an aspect, the invention provides a method of detecting an association between a genotype and an insulin resistance phenotype, by genotyping at least one IRM gene in a first population of known insulin resistance status; genotyping said IRM gene in a second population of known insulin resistance status; and determining whether a statistically significant association exists between the genotype and the phenotype.

In an aspect, the invention provides a method of estimating the frequency of a haplotype for a set of IRM polymorphisms in a population by genotyping at least a first IRM gene in the population; genotyping a second, different, IRM gene in the population, determining the identity of polymorphisms in each IRM gene, and applying an haplotype determination method to the identities of the nucleotides determined to obtain an estimate of said frequency. As used herein, the term "haplotype determination method" is used to refer to all methods for determining haplotypes known in the art including expectation-maximization algorithms (see, e.g., U.S. patent no. 6,346,381, Lange K., Mathematical and Statistical Methods for Genetic Analysis, Springer, New York, 1997; Weir, B. S., Genetic data Analysis II: Methods for Discrete population genetic Data, Sinauer Assoc., Inc., Sunderland, Mass., USA, 1996; ) Preferably, maximum-likelihood haplotype frequencies are computed using an Expectation-Maximization (EM) algorithm (see Dempster et al., J. R. Stat. Soc., 39B:1-38, 1977; Excoffier L. and Slatkin M., Mol. Biol. Evol., 12(5): 921-927, 1995) which can be carried out using computer implemented methods, for example the EM-HAPLO program (Hawley M. E. et al., Am. J. Phys. Anthropol., 18:104, 1994) or the Arlequin program (Schneider et al., Arlequin: a software for population genetics data analysis, University of Geneva, 1997).

In a related aspect, the invention provides a method of detecting an association between a haplotype and a phenotype by estimating the frequency of at least one haplotype in a population with a first phenotype (e.g., eIS) as described above, estimating the frequency of said haplotype in a population with a second phenotype (e.g., eIR) as described above, and determining whether a statistically significant association exists between said haplotype and said phenotype. In an embodiment, the haplotype exhibits a p-value of 0.001 in an association with a eIR phenotype or an eIS phenotype.

V.SCREENING FOR MODULATORS OF IRM EXPRESSION AND ACTIVITY The present invention provides screening methods to identify agents useful for the treatment of IR and IR-related conditions. The screening methods generally involve conducting various types of assays to identify agents that modulate the expression or activity of an IRM gene product. A number of different screening protocols can be utilized to identify agents that modulate the level of expression of IRM in cells, particularly mammalian cells, especially human cells. In general terms, the screening methods involve screening a plurality of agents to identify an agent that changes the activity of IRM by binding to an IRM polypeptide, preventing an inhibitor from binding to an IRM polypeptide, or activating or inhibiting expression of IRM, for example.

As used herein, a "modulator"of IRM activity or expression may inhibit or stimulate expression of an IRM gene product. Thus, in one embodiment, the administration of the modulator reduces expression or activity of the IRM gene product in the cell or animal (e.g., it acts as an antagonist or inhibitor). In a different embodiment, the administration of the modulator increases expression or activity of the IRM gene product in the cell or animal (e.g., it acts as an agonist or stimulator).

Modulators and/or active analogs identified in screening assays are formulated into pharmaceutical compositions effective in treating IR and related conditions.

IRM Polypeptide Binding and Interaction AssaysPreliminary screens can be conducted by screening for compounds capable of binding to IRM, as at least some of the compounds so identified are likely IRM modulators. Lead compounds identified during these screens can serve as the basis for the synthesis of more active analogs. Thus, in one aspect, the invention provides a method of screening for an agent to determine its usefulness in treating insulin resistance or a related condition by (a) contacting a polypeptide encoded by an IRM gene, or a cell expressing such a polypeptide with a test compound, and (b) determining whether the polypeptide binds to the test compound. Such binding is an indication that the test agent is useful in treatment of insulin resistance or a related condition. The binding assays usually involve contacting an IRM polypeptide with one or more test compounds and allowing sufficient time for the protein and test compounds to form a binding complex. Determining the ability of the test compound to directly bind to a IRM gene product can be accomplished, for example, by coupling the compound with a radioisotope or enzymatic label such that binding of the compound to the IRM gene product can be determined by detecting the labeled IRM gene product compound in a complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., E.C. Hulme, 1992, "Receptor-Ligand Interactions"in A Practical Approach/The Practical Approach Series (Series Eds D. Rickwood and BD Hames) IRL Press at Oxford University Press). The IRM polypeptide utilized in such assays can be purified or recombinant.

Assays for test compounds that modulate the activity of a IRM gene product or a biologically active portion thereof are also contemplated. The IRM gene products can, in vivo, interact with one or more cellular and extracellular molecules (such as, without limitation, peptides, proteins, hormones, cofactors and nucleic acids) hereinreferred to as "binding partners."Methods are known for identify its natural in vivo binding partners of IRMs, e.g., two and three-hybrid assays (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300). Such IRM gene product binding partners may be involved in the propagation of signals by the IRM gene product or downstream elements of a IRM gene product-mediated signaling pathway, or, alternatively, may be found to be inhibitors of the IRM gene product.

Assays may be devised through the use of the invention to identify compounds that modulate (e.g., affect either positively or negatively) interactions between a IRM gene product and its binding partners. Typically, the assay for compounds that interfere with the interaction between the IRM gene product and its binding partner involves preparing a reaction mixture containing the IRM gene product and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the IRM gene product and its binding partner. The assay for compounds that interfere with the interaction of the IRM gene product with its binding partner may be conducted in solution or in a format in which either the IRM gene product or its binding partner is anchored onto a solid surface or matrix Also within the scope of the present invention are methods for direct detection of interactions between the IRM gene product and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103).

Expression and Activity AssaysCertain screening methods involve screening for a compound that modulates (e.g., up-regulates or down-regulates) the expression or activity of an IRM in a cell. Such methods generally involve conducting cell-based assays in which test compounds are contacted with one or more cells expressing IRM and then detecting a change in IRM expression (e.g., levels of IRM RNA). In one embodiment, an assay for identification of modulators comprises contacting one or more cells (i.e., "test cells") with a test compound, and determining whether the test compound affects expression or activity of an IRM gene product in the cell. In an embodiment, the invention provides a method of screening for an agent to determine its usefulness in treating insulin resistance or a related condition by providing a cell expressing at least one insulin resistance marker (IRM) listed in Table 1; contacting the cell with a test agent; and determining whether the level of expression of an IRM is changed in the presence of the test agent, wherein a change is an indication that the test agent is useful in treatment of insulin resistance. Usually this determination comprises comparing the activity or expression in the test cell compared to a similar cell or cells (i.e., control cells) that have not been contacted with the test compound. Alternatively, cell extracts may be used in place of intact cells. In a related embodiment, the test compound is administered to a multicellular organism (e.g., a plant or animal). The IRM component may be wholly endogenous to the cell or multicellular organism or may be a recombinant cell or transgenic organism comprising one or more recombinantly expressed IRM gene products.

Generally, the effect of a test agent on the level of expression of an IRM RNA is determined. However, in other embodiments, the invention provides a method of screening for an agent to determine its usefulness in treating insulin resistance by (a) providing a composition comprising an IRM protein, or a cell expressing such a protein, with a test compound, (b) contacting the composition with a test agent and (c) determining whether the activity of the IRM protein is changed in the presence of the test product. A change is an indication that the test agent is useful in treating insulin resistance. In one aspect, the invention provides a method of screening for an agent to determine its usefulness in treating insulin resistance by (a) contacting a protein encoded by an IRM gene, or a cell expressing such a protein, with a test compound, wherein said polypeptide has a detectable biological activity; and (b) determining whether the level of biological activity of the protein is changed in the presence of the test agent, where a change is an indication that the test agent is useful in treatment of insulin resistance.

The assays can be carried out using any cell type that expresses a IRM gene including, in various embodiments, a cultured cell (e.g., a cell in a primary culture or an established cell line) and a cell in vivo. Preferably the cell expresses more than one IRM gene, e.g., at least about 3, at least about 5 or at least about 10 IRM genes. Exemplary cells include EBV-transfomed B-lymphocytes, well-known insulin-responsive cell lines such as 3T3-L1 adipocytes, CHO, and L6 rat skeletal myotubes. Other cell lines, such as mouse macrophage RAW cell line, Jurkat cells (acute leukemic T-cell), PC12 cells (rat neuronal), Hela cells, and HepG2 cells may also be used if the desired IRMs are also expressed at a detectable level in these cells. Similarly cell lines or primary cultures from patients with Burkitt's lymphoma, B-cell prolymphocytic leukemia (B-PLL), B-cell chronic lymphoblastic leukemia (B-CLL), and B-cell acute lymphoblastic leukemia (B-ALL) can be used (e.g., Burkitt's lymphoma cell lines (Raji, Daudi), B-PLL line (p11A-1-1), and B-ALL lines (MOLT-3, MOLT-4)). Many other suitable cells or cell lines will be known to the practicioner.

In one embodiment, the cell type is a cell in cell culture, such as a stably transformed cell line. As noted, EBV-transfomed B-lymphocytes can be used. Transformed B-lymphocytes can be prepared using well known techniques. According to one method, for example, a whole blood sample (12-15 ml) is collected in citrate (yellow top) or heparin (green top) vacutainer tube. Isolation of lymphocytes is performed using a one-step centrifugation technique developed by Boyum, 1964, Nature 204:793. The centrifugation solution (IsoPrep, and Red-Out) used to isolate the lymphocytes is purchased from Robbins Scientific Corp (Cat #1070-03-0, and Cat#1069-01-0). The isolated blood lymphocytes are cultured in tissue culture medium RPMI 1640 supplemented with 10% fetal bovine serum and essential amino acids. The culture is infected with EBV supernatant using a protocol developed by Henderson et al., 1977, Virology 76:152-63. The cells usually starts showing morphological changes after 3 to 4 days when dividing cells can be seen as dumbbell shaped structures under an inverted microscope. Typical morphological changes manifested by an actively growing cell culture comprise cellular clumps which can be seen with a naked eye. Usually it takes six to eight weeks to obtain a fully transformed culture showing typical manifestation of big cellular masses.

In one embodiment, cell lines are prepared using cells from a subject of known insulin resistance status, e.g., an individual with an eIR phenotype or a eIS phenotype, for example. Cell lines prepared from eIR phenotype subjects are referred to as "eIR cell lines." Such cell lines from B-cells can be called "eIR B-cell lines." Cell lines prepared from eIS phenotype subjects are referred to as "eIS cell lines." Such cell lines from B-cells can be called "eIS B-cell lines."It will be recognized that, although the cells used often are human cells, animal cells can be used (e.g., expression of nonhuman homologs of human IRM genes can be monitored, or expression of human IRM genes in IRM gene transgenic animals such as mice can be monitored). When nonhuman cells are used it is often desirable to use nucleic acid or antibody probes that recognize the nonhuman homologs of the human IRM genes (e.g. usually detectable using a probe based on the human IRM sequence). One of ordinary skill in the art will be able to identify such homologs and obtain suitable probes based on the information in Table 1. In one embodiment, the test agent is administered to an animal and the effect of the agent on expression of an IRM homolog in a tissue of the animal (e.g., blood or a blood fraction) is detected.

IRM expression by cells can be detected in a number of different ways including the methods described supra in the context of diagnostic methods. As described supra, the expression level of IRM in a cell can be determined by isolating RNA from the cell and probing the mRNA expressed in a cell with a probe that specifically hybridizes with a transcript (or complementary nucleic acid derived therefrom) of IRM. Alternatively, IRM protein can be detected using immunological methods in which a cell lysate is probe with antibodies that specifically bind to an IRM polypeptide. Alternatively, the level of activity of an IRM polypeptide can be determinedThe effect of an agent on IRM gene expression in a cell or in vitro system can be compared to a baseline value, which is typically the level of expression by the cell or in vitro system in the absence of the test agent. Expression levels can also be determined for cells that do not express IRM as a negative control. Such cells generally are otherwise substantially genetically the same as the test cells. In other embodiments, the baseline value can be a value for a control sample or a statistical value that is representative of IRM expression levels for a control population (e.g., healthy individuals not at high risk for IR).

As noted supra, the invention provides drug screening assays in which the expression level of more than one IRM gene is monitored. Monitoring expression of multiple genes provides for more robust assays. Thus, in various embodiments, the effect of a agent on expression of a combination of IRM genes (e.g., at least 2, 3, 4, 5, 6, 7, 8, 9, 10, 11, 12, 15, 20, or 25 or more of the IRMs listed in Tableor selected from a subpanel of the IRMs disclosed herein) are determined. In general, an agent that changes expression of multiple IRM genes on a panel of particular interest as a drug candidate or lead drug. Devices comprising arrays of probes for specific IRM gene products, e.g., as described herein, may be used to conduct the assays. As described below, an agent identified in a screening assay described herein may be administered to a test animal (e.g., primates, dogs, rabbits, rodents, e.g., mice) to determine the animal"s response to the agent (e.g., whether the animal"s response to insulin is affected by the agent).

It is also possible to use cells that are stably or transiently transfected with a vector or expression cassette having a nucleic acid sequence which encodes the IRM protein. The cells are maintained under conditions appropriate for expression of the protein and are contacted with a putative agent. Other cell-based assays are reporter assays conducted with cells that do not express IRM. Certain of these assays are conducted with a heterologous nucleic acid construct that includes a IRM promoter that is operably linked to a reporter gene that encodes a detectable product. IRM gene promoters are located, in most cases, within a region about 300 to 1000 bp upstream (or 5") of the transcription start sites. Certain IRM gene promoters are described in GenBank, which can be accessed via the internet at "http://www.ncbi.nlm.nih.gov/", and the scientific literature. A number of different reporter genes can be utilized. Exemplary reporters include green fluorescent protein, ß -glucuronidase, chloramphenicol acetyl transferase, luciferase, ß -galactosidase, alkaline phosphatase, and the like. In these assays, cells harboring the reporter construct are contacted with a test compound. A test compound that either activates the promoter by binding to it or triggers a cascade that produces a molecule that activates the promoter causes expression of the detectable reporter. A variety of different types of cells can be utilized in the reporter assays (e.g., eukaryotic cells such as yeast, COS, CHO, HepG2, and HeLa cell lines).

Transgenic AnimalsTransgenic animals expressing one or more IRM-encoding polynucleotides can also be used for drug screening and other methods of the invention. Suitable transgenic non-human multicellular organisms (e.g., plants and non-human animals) or unicellular organisms (e.g., yeast) comprising an exogenous IRM gene sequence (which may be a coding sequence or a regulatory sequence) nonhuman animals such as mice, rats, rabbits, monkeys, apes, and pigs. In one embodiment, the organism expresses an exogenous IRM polypeptide, having a sequence of a human IRM protein.

The invention also provides unicellular and multicellular organisms (or cells therefrom) in which a gene encoding a homolog of a human IRM is mutated or deleted (i.e., in a coding or regulatory region) such that native IRM protein is not expressed, or is expressed at reduced levels or with different activities when compared to wild-type cells or organisms. Such cells and organisms are often referred to as "gene knock-out" cells or organisms.

The invention further provides cells and organisms in which an endogenous IRM gene is either present or optionally mutated or deleted and an exogenous IRM gene or variant (e.g., human IRM ) is introduced and expressed. Cells and organisms of this type will be useful, for example, as model systems for identifying modulators of IRM activity or expression; determining the effects of mutations in the IRM gene on insulin resistance.

Methods for alteration or disruption of specific genes are well known to those of skill, see, e.g., Baudin et al., 1993, Nucl. Acids Res. 21:3329; Wach et al., 1994, Yeast 10:1793; Rothstein, 1991, Methods Enzymol.194:281; Anderson, 1995, Methods Cell Biol. 48:31; Pettitt et al., 1996, Development 122:4149-4157; Ramirez-Solis et al., 1993, Methods Enzymol. 225:855; and Thomas et al., 1987, Cell 51:503. Typically, such methods involve altering or replacing all or a portion of the regulatory sequences controlling expression of the particular gene to be regulated. The regulatory sequences, e.g., the native promoter can be altered. One conventional technique for targeted mutation of genes involves placing a genomic DNA fragment containing the gene of interest into a vector, followed by cloning of the two genomic arms associated with the targeted gene around a selectable neomycin-resistance cassette in a vector containing thymidine kinase. This "knock-out" construct is then transfected into the appropriate host cell, i.e., a mouse embryonic stem (ES) cell, which is subsequently subjected to positive selection (using G418, for example, to select for neomycin-resistance) and negative selection (using, for example, FIAU to exclude cells lacking thymidine kinase), allowing the selection of cells which have undergone homologous recombination with the knockout vector. This approach leads to inactivation of the gene of interest. See, e.g., U.S. patents 5,464,764; 5,631,153; 5,487,992; and, 5,627,059. "Knocking out" expression of an endogenous gene can also be accomplished by the use of homologous recombination to introduce a heterologous nucleic acid into the regulatory sequences (e.g., promoter) of the gene of interest. To prevent expression of functional enzyme or product, simple mutations that either alter the reading frame or disrupt the promoter can be suitable. To up-regulate expression, a native promoter can be substituted with a heterologous promoter that induces higher levels of transcription. Also, "gene trap insertion" can be used to disrupt a host gene, and mouse ES cells can be used to produce knockout transgenic animals, as described for example, in Holzschu (1997) Transgenic Res 6: 97-106. Other methods are known in the art.

Altering the expression of endogenous genes by homologous recombination can also be accomplished by using nucleic acid sequences comprising the structural gene in question. Upstream sequences are utilized for targeting heterologous recombination constructs. Utilizing structural gene sequence information, such as can be determined by reference to Table 1 and published materials (e.g., in GenBank) one of skill in the art can create homologous recombination constructs with only routine experimentation. Homologous recombination to alter expression of endogenous genes is described in, e.g., U.S. Patent 5,272,071, and WO 91/09955, WO 93/09222, WO 96/29411, WO 95/31560, WO 91/12650, and Moynahan, 1996, Hum. Mol. Genet. 5:875.

Test CompoundsThe screening methods can be conducted with essentially any type of compound potentially capable of modulating IRM expression. Consequently, test compounds can be of a variety of general types including, but not limited to small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries or combinatorial libraries, for example.

Combinatorial chemistry methodology can be used to create vast numbers of oligonucleotides (or other compounds) that can be rapidly screened for specific oligonucleotides (or compounds) that have appropriate binding affinities and specificities toward any target, such as the IRM proteins and genes described herein (for general background information Gold (1995) J. of Biol. Chem. 270:13581-13584). The creation and simultaneous screening of large libraries of synthetic molecules can be carried out using well-known techniques in combinatorial chemistry, for example, see van Breemen (1997) Anal Chem 69:2159-2164; Lam (1997) Anticancer Drug Des 12:145-167 (1997). Combinatorial libraries can be produced for many types of compound that can be synthesized in a step by step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbanates. A number of different types of combinatorial libraries and methods for preparing such libraries have been described, including for example, PCT publications WOWOWOWOand WOeach of which is incorporated herein by reference. Several methods of automating assays have been developed in recent years so as to permit screening of tens of thousands of compounds in a short period. See, e.g., Fodor et al., 1991, Science 251: 767-73, and other descriptions of chemical diversity libraries, which describe means for testing of binding affinity by a plurality of compounds. Peptide libraries can also be generated by phage display methods.

IRM Expression or Activity Modulators The invention further provides (i) novel agents identified by the above-described screening assays, (ii) pharmaceutical compositions comprising an agent identified by the above-described screening assay and (iii) methods for treating a subject who is insulin resistant, has an insulin resistance associated condition (e.g., diabetes), or is susceptible to insulin resistance or an insulin resistance associated condition by administering an agent identified by the above-described screening assays.

Compounds that are initially identified by any of the foregoing screening methods can be further tested to validate the apparent activity. Preferably such studies are conducted with suitable animal models. The basic format of such methods involves administering a lead compound identified during an initial screen to an animal that serves as a model for humans and then determining if the response to insulin (e.g., an effect on blood glucose levels after administration of insulin) is affected by administration of the agent. Examples of suitable animals include, but are not limited to mammals, primates, such as mice and rats. Exemplary animal models for insulin resistance and type II diabetes include Zucker diabetic-fatty (ZDF) rats, GK rats, Otsuka Long-Evans Tokushima Fatty (OLETF) rats, db/db mice, and BSB mice.

In one aspect, a method of preparing a medicament for use in treating insulin resistance or an IR related condition is provided. The method involves determining that an agent is useful for treatment of insulin resistance using an assay as described herein and formulating the agent for administration to a primate (e.g., human). For example, suitable formulations may be sterile and/or substantially isotonic and/or in full compliance with all Good Manufacturing Practice (GMP) regulations of the U.S. Food and Drug Administration and/or in a unit dosage form.

VI.DEVICES AND KITS FOR DIAGNOSTIC APPLICATIONS Devices and reagents useful for diagnostic, prognostic, drug screening, and other methods are provided. In one aspect, a device comprising immobilized probe(s) specific for one or more IRM gene products (polynucleotides or proteins) is provided. The probes can bind polynucleotides (e.g., based on hybridization to IRM RNA or cDNA) or polypeptides (e.g., based on specific binding to an IRM polypeptide).

In one embodiment, an array format is used in which a plurality (at least 2, usually at least 3 or more) of different probes are immobilized. The term "array" is used in its usual sense and means that each of a plurality of probes, usually immobilized on a substrate, has a defined location (address) e.g., on the substrate. The number of probes on the array can vary depending on the nature and use of the device. For example, a dipstick format array for detecting IRM expression can have as few as 2 distinct probes, although usually more than 2 probes, and often many more, will be present. As noted, it is also contemplated that, in some embodiments, a device comprising a single immobilized probe can be used, although such a device taken by itself is generally not called an "array."A variety of binding and hybridization formats are known, including oligonucleotide arrays, cDNA arrays, dip sticks, pins, chips, or beads, southern, northern, dot and slot blots. Thus a device comprising a probe for an IRM gene product immobilized on a solid substrate is contemplated. Any of a variety of solid supports can be used, which may be made from glass (e.g., glass slides), plastic (e.g., polypropylene, nylon), polyacrylamide, nitrocellulose, or other materials. One method for attaching the nucleic acids to a surface is by printing on glass plates, as is described generally by Schena et al., 1995, Science 270:467-470; Shalon et al., 1996, Genome Res. 6:639-645. Another method for making microarrays is by making high-density oligonucleotide arrays. See, Fodor et al., 1991, Science 251:767-73; Lockhart et al., 1996, Nature Biotech 14:1675; and U.S. Pat. Nos. 5,578,832; 5,556,752; and 5,510,270).

It is contemplated that, in some embodiments, the substrate on which the probes are immobilized (e.g., chip or slide) includes a plurality of probes that are specific for IRM (e.g., in contrast to a chip or slide containing probes for all genes expressed in an organism, cell or tissue). For example, an array can be specifically designed based on the teachings herein to include probes to at least 2, at least 3, at least 4, at least 5, at least 6, or at least 10 insulin resistant markers disclosed herein. Thus, in an embodiment, at least about 10%, and sometimes at least about 25% or even at least about 50% of the immobilized probes on a device or array specifically bind (e.g., hybridize to) IRM gene products.

In one embodiment, the substrate comprises fewer than about 4000 distinct probes, often fewer than about 1000, fewer than about 100 distinct probes, fewer than about 50 distinct probes, fewer than about 10 distinct probes, fewer than about 5 distinct probes or fewer than about 3 distinct probes. As used in this context, a probe is "distinct"from a second probe if the two probes do not specifically bind the same polypeptide or polynucleotide (i.e., such as cDNA probes for different genes). In one embodiment, the probes are selected from monoclonal antibodies or other specific binding proteins (e.g., antibody derivatives or fragments) that specifically bind an IRM protein. Probes for polypeptides can also be immobilized in an array format, for example, in an ELISA format in multiwell plates. Also contemplated are kits comprising reagents for assessing expression of one or more IRM genes, such as probes and/or primers for detection or amplification of IRM gene products. In one embodiment, the probes are nucleic acid probes that specifically bind to a polynucleotide transcribed from an IRM gene. In an embodiment, the kit contains probes specific for a plurality (at least 2, preferably 3, often 4, sometime 5 or more) different IRM gene products (such as binding or hybridization targets for 1, 2, 3, 4, 5 or more IRMs selected from a panel of IRMs as described elsewhereherein). In one embodiment, the probes are selected from polynucleotides that specifically hybridize to IRM polynucleotides disclosed herein. Suitable reagents for binding with a nucleic acid (e.g. an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, and the like. Such reagents can be used, for example, to facilitate contemporaneous detection of multiple IRMs in a patient sample. The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, instructions for carrying out the detection methods of the invention, and calibration curves can also be included, a reference sample (or protein or nucleic acid) for calibration or comparison to expression levels determined for an individual, or reference values for IRM expression in normal and nonnormal populations in printed or electronic form.VII.METHODS OF TREATING INSULIN RESISTANCE AND RELATED CONDITIONS OR DISEASESIn another aspect, the present invention provides methods of treating insulin resistance or related conditions (e.g., typediabetes) by administering to a subject having or at risk for such a disease or condition, a therapeutically effective amount of an modulator of IRM function, e.g., a agonist (stimulator) or antagonist (inhibitor) or IRM function or gene expression. For inhibition of IRM function, Exemplary modulators include small molecule antagonists of (i.e., molecular weight less than 5000 Daltons, usually less than 3000 Daltons, often less than 500 Daltons, e.g., nucleic acids, peptides, carbohydrates, lipids, organic or inorganic molecule); anti-IRM binding agents (e.g., anti-IRM monoclonal antibodies); polypeptide inhibitors (e.g., dominant-negative mutants of IRMs); polynucleotide inhibitors (e.g., antisense, ribozyme and triplex polynucleotides); gene therapy (e.g., gene knockout); and the like. For stimulation of IRM function, exemplary modulators include small molecule agonists of IRM function and IRM polypeptides (which may be administered, e.g., in the form of polypeptides or nucleic acid expression vectors); and the like. Depending upon the individual"s condition, the agent can be administered in a therapeutic or prophylactic amount.

In one embodiment, for illustration and not limitation, an agent that increases activity or expression of an IRM that is downregulated (i.e., expressed at lower levels) in the eIR population compared to the eIS population is administered to treat insulin resistance or an insulin resistance-related condition. In a different embodiment, for illustration and not limitation, an agent that decreases activity or expression of an IRM that is upregulated (i.e., expressed at higher levels) in the eIR population compared to the eIS population is administered to treat insulin resistance or an insulin resistance-related condition.

In one aspect, the therapeutic methods of the invention make use of agents or pharmaceuticals known or believed to modulate expression or activity of an IRM described herein, but not previously recognized as having an effect on insulin resistance. In a related aspect, the agent is not previously recognized as having an effect on one or more insulin resistance-related conditions.

The methods and reagents of the invention may be used in treatment of animals such as mammals (e.g., humans, non-human primates, cows, sheep, goats, horses, dogs, cats, rabbits, rats, mice) or in animal or in vitro (e.g., cell-culture) models of human diseases.

Methods for Inhibiting IRM ExpressionA variety of ways to reduce expression or activity of an IRM are known in the art. In one embodiment, an inhibitory polynucleotide is administered. Examples of inhibitory polynucleotides include antisense, triplex, and ribozyme reagents that target or hybridize to IRM polynucleotides. Some therapeutic methods of the invention involve the administration of an oligonucleotide that functions to inhibit IRM activity under in vivo physiological conditions, and is relatively stable under those conditions for a period of time sufficient for a therapeutic effect. Polynucleotides can be modified to impart such stability and to facilitate targeting delivery of the oligonucleotide to the desired tissue, organ, or cell.

Antisense Polynucleotides According to the invention, antisense oligonucleotides and polynucleotides are used to inhibit expression of an IRM gene. Antisense polynucleotides useful in the present invention comprise an antisense sequence of at least about 10 bases, typically at least 12 or 14, and up to about 1000 contiguous nucleotides or more that specifically hybridize to a sequence from mRNA transcribed from the IRM gene. More often, the antisense polynucleotide of the invention is from about 12 to about 50 nucleotides in length or from about 15 to about 25 nucleotides in length. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors.

Generally, to assure specific hybridization, the antisense sequence is substantially complementary to the target IRM mRNA sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to IRM RNA or its gene is retained as a functional property of the polynucleotide.

In one embodiment, the antisense sequence is complementary to relatively accessible sequences of the IRM mRNA (e.g., relatively devoid of secondary structure). This can be determined by analyzing predicted RNA secondary structures using, for example, the MFOLD program (Genetics Computer Group, Madison WI) and testing in vitro or in vivo as is known in the art. Another useful method for identifying effective antisense compositions uses combinatorial arrays of oligonucleotides (see, e.g., Milner et al., 1997, Nature Biotechnology 15:537).

The antisense nucleic acids (DNA, RNA, modified, analogues, and the like) can be made using any suitable method for producing a nucleic acid, such as the chemical synthesis and recombinant methods disclosed herein. In one embodiment, for example, antisense RNA molecules of the invention may be prepared by de novo chemical synthesis. Alternatively, an antisense RNA that hybridizes to IRM mRNA can be made by inserting (ligating) an IRM DNA sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention. The antisense oligonucleotides of the invention can be used to inhibit IRM activity in cell-free extracts, cells, and animals, including mammals and humans. In one embodiment, the antisense oligonucleotide inhibits expression of the IRM in a test cell line by at least about 25%, preferably at least about 50%, compared to no treatment. The test cell line is typically a an established human cell line (i.e., available from the ATCC, or prepared by EBV transformation of a leukocyte cell as described herein).

For general methods relating to antisense polynucleotides, see D.A. Melton, Ed., 1988, Antisense RNA AND DNA Cold Spring Harbor Laboratory, Cold Spring Harbor, NY. See also, Dagle et al., 1991, Nucleic Acids Research, 19:1805.

Triplex Oligo- and PolynucleotidesThe present invention provides oligo- and polynucleotides (e.g., DNA, RNA, PNA or the like) that bind to double-stranded or duplex IRM nucleic acids (e.g., in a folded region of the IRM RNA or in the IRM gene), forming a triple helix-containing, or "triplex" nucleic acid. Triple helix formation results in inhibition of IRM expression by, for example, preventing transcription of the IRM gene, thus reducing or eliminating IRM activity in a cell. Without intending to be bound by any particular mechanism, it is believed that triple helix pairing compromises the ability of the double helix to open sufficiently for the binding of polymerases, transcription factors, or regulatory molecules to occur.

Triplex oligo- and polynucleotides of the invention are constructed using the base-pairing rules of triple helix formation (see, e.g., Cheng et al., 1988, J Biol. Chem. 263: 15110; Ferrin and Camerini-Otero, 1991, Science 354:1494; Ramdas et, 1989, J. Biol. Chem. 264:17395; Strobel et al., 1991, Science 254:1639; and Rigas et al., 1986, Proc. Natl. Acad. Sci. U.S.A. 83:9591; and the IRM mRNA and/or gene sequence. Typically, the triplex-forming oligonucleotides of the invention comprise a specific sequence of from about 10 to at least about 25 nucleotides or longer "complementary" to a specific sequence in the IRM RNA or gene (i.e., large enough to form a stable triple helix, but small enough, depending on the mode of delivery, to administer in vivo, if desired). In this context, "complementary"means able to form a stable triple helix.

RibozymesThe present invention also provides ribozymes useful for inhibition of IRM activity. The ribozymes of the invention bind and specifically cleave and inactivate IRM mRNA. Useful ribozymes can comprise 5"- and 3"-terminal sequences complementary to the IRM mRNA and can be engineered by one of skill on the basis of the IRM mRNA sequence disclosed herein (see PCT publication WO 93/23572, supra). Ribozymes of the invention include those having characteristics of group I intron ribozymes (Cech, 1995, Biotechnology 13:323) and others of hammerhead ribozymes (Edgington, 1992, Biotechnology 10:256).

Ribozymes of the. invention include those having cleavage sites such as GUA, GUU and GUC. Other optimum cleavage sites for ribozyme-mediated inhibition of IRM activity in accordance with the present invention include those described in PCT publications WO 94/02595 and WO 93/23569. Short RNA oligonucleotides between 15 and 20 ribonucleotides in length corresponding to the region of the target IRM gene containing the cleavage site can be evaluated for secondary structural features that may render the oligonucleotide more desirable. The suitability of cleavage sites may also be evaluated by testing accessibility to hybridization with complementary oligonucleotides using ribonuclease protection assays, or by testing for in vitro ribozyme activity in accordance with standard procedures known in the art. In one embodiment, the ribozymes of the invention are generated in vitro and introduced into a cell or patient. In another embodiment, gene therapy methods are used for expression of ribozymes in a target cell ex vivo or in.

Administration of OligonucleotidesTypically, the therapeutic methods of the invention involve the administration of an oligonucleotide that functions to inhibit or stimulate IRM activity under in vivo physiological conditions, and is relatively stable under those conditions for a period of time sufficient for a therapeutic effect. As noted above, modified nucleic acids may be useful in imparting such stability, as well as for targeting delivery of the oligonucleotide to the desired tissue, organ, or cell.

Oligo- and poly-nucleotides can be delivered directly as a drug in a suitable pharmaceutical formulation, or indirectly by means of introducing a nucleic acid into a cell, including liposomes, immunoliposomes, ballistics, direct uptake into cells, and the like as described herein. For treatment of disease, the oligonucleotides of the invention will be administered to a patient in a therapeutically effective amount. A therapeutically effective amount is an amount sufficient to ameliorate the symptoms of the disease or modulate IRM activity in the target cell. Methods useful for delivery of oligonucleotides for therapeutic purposes are described in U.S. Patent 5,272,065. In another embodiment, oligo- and poly-nucleotides can be delivered using gene therapy and recombinant DNA expression plasmids.

AntibodiesIn one aspect of the invention, antibodies, e.g., monoclonal antibodies, that specifically bind IRM polypeptides antibodies are used to inhibit IRM activity in treatment of IR or IR-related conditions. As discussed above, anti-IRM antibodies are also used in the diagnostic and prognostic methods of the invention. The antibodies of the invention will specifically recognize and bind polypeptides which have an amino acid sequence identical, or substantially identical, to the amino acid sequence of the IRMs described herein, or an immunogenic fragment thereof. The antibodies of the invention usually exhibit a specific binding affinity of at least about 10⁷, 10⁸, 10⁹, or 10¹⁰M⁻¹.

Anti-IRM antibodies can be made by a variety of means well known to those of skill in the art. Methods for production of polyclonal or monoclonal antibodies are well known in the art. See, e.g., Supra Kohler and Milstein, 1975, Nature 256:495-97; and Harlow and Lane. These techniques include antibody preparation by selection of antibodies from libraries of recombinant antibodies in phage or similar vectors. See, Huse et al., 1989, Science 246:1275-81; and Ward et al., 1989, Nature 341:544-46.

For production of polyclonal, antibodies, an appropriate target immune system is selected, typically a mouse or rabbit, but also including goats, sheep, cows, chickens, guinea pigs, monkeys and rats. The immunoglobulins produced by the host can be precipitated, isolated and purified by routine methods, including affinity purification. Substantially monospecific antibody populations can be produced by chromatographic purification of polyclonal sera.

In some embodiments of the invention, anti-IRM monoclonal antibodies are humanized, human or chimeric, in order to reduce their potential antigenicity, without reducing their affinity for their target. Humanized antibodies have been described in the art. See, e.g., Queen, et al., 1989, Proc. Nat"l Acad. Sci. USA 86:10029; U.S. Patent Nos. 5,563,762; 5,693,761; 5,585,089 and 5,530,101. The human antibody sequences used for humanization can be the sequences of naturally occurring human antibodies or can be consensus sequences of several human antibodies. See Kettleborough et al., Protein Engineering 4:773 (1991); Kolbinger et al., Protein Engineering 6:971 (1993).

Humanized monoclonal antibodies against IRMs can also be produced using transgenic animals having elements of a human immune system (see, e.g., U.S. Patent Nos. 5,569,825; 5,545,806; 5,693,762; 5,693,761; and 5,7124,350).

Useful anti-IRM binding compositions can also be produced using phage display technology (see, e.g., Dower et al., WO 91/17271 and McCafferty et al., WO In these methods, libraries of phage are produced in which members display different antibodies on their outer surfaces. Antibodies are usually displayed as Fv or Fab fragments. Phage displaying antibodies with a desired specificity are selected by affinity enrichment to an IRM polypeptide.

An antibody (e.g. an anti-IRM antibody), is substantially pure when at least about 80%, more often at least about 90%, even more often at least about 95%, most often at least about 99% or more of the polypeptide molecules present in a preparation specifically bind the same antigen (e.g., IRM polypeptide). For pharmaceutical uses, anti-IRM immunoglobulins of at least about 90 to 95% homogeneity are preferred, and 98 to 99% or more homogeneity are most preferred.

The antibodies of the present invention can be used with or without modification. Frequently, the antibodies will be labeled by joining, either covalently or non-covalently, a substance which provides for a detectable signal. Such labels include those that are well known in the art, e.g., radioactive, fluorescent, or bioactive (e.g., enzymatic) labels. As labeled binding entities, the antibodies of the invention may be particularly useful in diagnostic applications.

Methods for Increasing IRM Gene Product LevelsGene therapy approaches can be used to increase IRM expression. Such methods generally involve administering to an individual a nucleic acid molecule that encodes IRM polypeptide or an active fragment thereof. The administered nucleic acid increases the level of IRM expression in one or more tissues. The nucleic acid is administered to achieve synthesis of IRM in an amount effective to obtain a therapeutic or prophylactic effect in the individual receiving the therapy. As used herein, the term "gene therapy" refers to therapies in which a lasting effect is obtained with a single treatment, and methods wherein the gene therapeutic agents are administered multiple times to achieve or maintain the desired increase in IRM expression.

The nucleic acid molecules encoding IRM can be administered ex vivo or in vivo. Ex vivo gene therapy methods involve administering the nucleic acid to cells in vitro and then transplanting the cells containing the introduced nucleic acid back into the individual being treated. Techniques suitable for the in vitro transfer of IRM nucleic acids into mammalian cells include, but are not limited to, the use of liposomes, electroporation, microinjection, cell fusion, DEAE-dextran and calcium phosphate precipitation methods. Once the cells have been transfected, they are subsequently introduced into the patient.

In vivo gene therapy methods involve the direct administration of nucleic acid or a nucleic acid/protein complex into the individual being treated. In vivo administration can be accomplished according to a number of established techniques including, but not limited to, injection of naked nucleic; acid, viral infection, transport via liposomes and transport by endocytosis. Of these, transfection with viral vectors and viral coat protein-liposome mediated transfection are commonly used methods (see, e.g., Dzau et al., 1993, Trends in Biotechnology 11:205-210). Suitable viral vectors include, for example, adenovirus, adeno-associated virus and retrovirus vectors.

In a related aspect, levels of an IRM polypeptide are increased in a cell or patient by administration of an IRM polypeptide. The polypeptide can be prepared using routine recombinant techniques. Alternatively, the polypeptide can be prepared by purification according to method known in the art.

Pharmaceutical Compositions, Dosage & AdministrationThe present invention further provides therapeutic compositions comprising agonists, antagonists, or ligands of IRMs.. The therapeutic compositions can be directly administered under sterile conditions to the host to be treated. However, while it is possible for the active ingredient to be administered alone, it is often preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious to the patient. For example, the bioactive agent can be complexed with carrier proteins such as ovalbumin or serum albumin prior to their administration in order to enhance stability or pharmacological properties such as half-life.

Therapeutic formulations can be prepared by any methods well known in the art of pharmacy. See, e.g., Gilman et al. (eds.), 1990, Goodman and Gilman"s: The Pharmacological Bases of Therapeutics (8th ed.) Pergamon Press; and (1990) Remington"s Pharmaceutical Sciences (17th ed.) Mack Publishing Co., Easton, PA.; Avis et al (eds.) (1993) Pharmaceutical Dosage Forms: Parenteral Medications Dekker, N.Y.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering. a composition containing a pharmaceutically acceptable carrier via oral, intranasal, rectal, topical, intraperitoneal, intravenous, intramuscular, subcutaneous, subdermal, transdermal, intrathecal, and intracranial methods.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

VIII.METHOD FOR IDENTIFYING GENE SEQUENCES ASSOCIATED WITH A DISEASE OR CONDITIONSIn a different aspect, the invention provides a method for identifying a gene or plurality of genes whose expression level is associated with a disease state or medical condition (hereinafter "disease"). The genes so identified, including corresponding gene products, are targets for intervention to prevent or treat the disease, are useful for diagnosis or prognosis of the disease (e.g., by detection of a gene expression pattern diagnostic of a disease state), may be used as targets for drug screening for agents useful for treatment of the disease, and many other uses.

In one embodiment of the invention, the method involves identifying a first population of human subjects, where the subjects suffer from, or are at high risk of, developing the disease, and identifying a second population of human subjects, where the subjects are at low risk of developing the disease. The method can be used for any disease for which a population suffering from, or with high susceptibility to, the disease can be distinguished from a population not suffering from, or with relatively low susceptibility to the disease. Examples of such diseases include insulin resistance and IR associated diseases (e.g., Type 2 diabetes), cardiovascular disease disease including dyslipidemia (e.g. high levels of fasting LDL and /or triglyceride, or low levels of fasting HDL), Atherosclerosis-related events including myocardial infarction, restenosis, cerebro-vascular disease and peripheral vascular disease. Other examples may include autoimmune disorders such as rheumatoid arthritis and allergy.

In an embodiment, the first and second populations each comprise at least 3, often at least 5, and sometimes at least 10 individuals. In some embodiments, the individuals are matched for age, sex , ethnicity and/or other clinically relevant criteria.

Age- and gender-match refers to the process of matching the first study population (e.g. eIR group which is often called the case group) and the second study group(e.g. eIS group which is often called the control group) during the initial selection of study subjects. Age-matched groups refer to the mean age of the case group is similar (i.e. not significantly different) from the mean age of the control group, as determined by a standard chi-square test with a p-value >0.05. Gender-matched groups mean the numbers of male and/or female or the ratio of male/female for the case and control groups are identical or similar. The similarity (or non-significant difference) can be determined by a standard chi-square test with a p-value >0.05.

In addition to age-and gender-matched, ethnicity match is an important process required in all genetic studies. For a relatively homogenous population such as Taiwan-Chinese, this is accomplished by selecting the case and control individuals from the same city or province. For a heterogeneous population such as the US population, there are in general five major ethnic groups: European-Americans, African-Americans, Mexican-Americans, Native Americans, and Asian-Americans. Ethnicity match in this case often refers to selecting one of the five ethnic groups to be used for both case and control groups in the study.

Cells are obtained from each of the populations and genes that are differentially expressed in the cells of the first population and the second population are identified. In an embodiment, the cell from a tissue of each individual are used to establish a cell line, e.g., an immortalized cell line, e.g., an immortalized B cell line, and genes are identified that are expressed at a higher level in the cell lines of one population compared to the other. In one embodiment the cell lines are derived by immortalization of blood cells from the individuals. In one embodiment, the cell lines are immortalized B-lymphocytes. Methods for establishing cell lines from blood lymphocytes are well known, and include, for example, EBV-mediated transformation. See, e.g., Henderson et al., 1977, Virology 76:152-63. As noted, EBV-transfomed B-lymphocytes can be prepared from isolated blood lymphocytes by infection with EBV supernatant and culturing the cells for six to eight weeks to obtain a fully transformed culture.

To identify genes that are differentially expressed in the two populations, any of a variety of methods can be used. Usually, RNA is isolated from the cell lines and probes are made from the RNA. In an embodiment, the RNA (or corresponding cDNA or other probes) of the cell lines for individuals in each population are pooled. For example, the RNA from each cell line can be pooled (in equal amounts from each individual cell line) before labeling. Alternatively, labeled probes corresponding to several cell lines can be mixed after labeling. Usually, the probes corresponding to each population are differently labeled so that they can be distinguished.

The optionally pooled probes (e.g., cDNA, RNA etc) are used in routine methods to identify genes that are differentially expressed in a tissue. See, e.g., Lockhart et al., 1996, Nature Biotech 14:1675; U.S. Pat. Nos. 5,578,832; 5,556,752; 5,510,270; Schena et al., 1995, Science 270:467-70 In one embodiment, the tissue is blood, e.g., blood lymphocytes. One method for identification of is by hybridization or the probes to arrayed oligonucleotide or cDNA sequences (e.g., expressed sequence tags) as described in the Examples, infra (e.g., by hybridizing the pooled probe to a nucleic acid array comprising > 100 expressed sequence tags from the tissue).

Thus, in one embodiment, gene sequences associated with a disease are identified by identifying a first population of human subjects who suffer from or are at increase risk of developing a disease, identifying a second population of human subjects at low risk of developing the disease and identifying RNA sequences differentially expressed in the first population compared to the second population. In an embodiment, the identifying steps include obtaining cell lines derived from a tissue from each of the subjects in the first and second populations, obtaining RNA from said cell lines, preparing an optionally pooled probe corresponding the RNA from each cell line (e.g., by pooling RNA prior to reverse transcription or pooling cDNA after reverse transcription), and hybridizing the pooled probe to a nucleic acid array comprising sequences expressed in a human tissue, such as blood.

Using this method, typically, at least 3 genes (RNA sequences) are determined to be differentially expressed in the first population compared to the second population.

In one illustrative embodiment, the first population is an extreme insulin resistant population (e.g., OGTT Glu at 120m > 140 mg/dl; SSPG mean > 250 mg/dl; OGTT Ins at 60m > 100 µ IU/ml Et; OGTT Ins at 120 m > 100 µ IU/ml) and the second population is an extreme insulin sensitive population (e.g., OGTT Glu at 120m < 100 SSPG mean < 120 mg/dl; OGTT Ins at 60m < 60 µIU/ml OR; OGTT Ins at 120 m < 40 µ IU/ml). In a second illustrative embodiment, the first population is an extreme high HDL population (e.g., fasting HDL > 60 mg/dl; age > 18 yr old; normal glucose tolerance test; non diabetic; no cardiovascular disease) and the second population is an extreme low HDL population (e.g., fasting HDL < 30 mg/dl; age > 18 yr old). In yet another illustrative embodiment, the first population is an extreme obese/high body mass (Body Mass Index > 30; age > 18 yr old; cell lines available) and the second population is an extreme lean/low body mass population (Body Mass Index (Kg/M²) < 20; age > 18 yr old; normal glucose tolerance test; non diabetic; no cardiovascular disease). Usually, the first population is age, gender and ethnicity matched with the second population.

IX.EXAMPLES The following examples are provided solely to illustrate in greater detail certain aspects of the invention and are not to be construed to limit the scope of the invention.

Example 1:Taiwan Insulin Resistance Family (TWIR) Study: Enrollment and Phenotype AnalysisThe TWIR families were collected via three ascertainment schemes: (1) both parents affected with NIDDM, (2) one parent affected with NIDDM, and (3) both parents clinically normal. This approach maximized the opportunity to identify linkages because IR segregates with high frequency in families with one or two affected parents. Some families with clinically normal parents were also included since IR also occurs in individuals without NIDDM.

A total of 112 Chinese nuclear families were collected at the Diabetes Clinics of Tri-service General Hospital in Taiwan between 1993 -1996. Among these, 81 families met selection criteria for enrollment into the linkage study: At least one sib pair per family if both parents available for study; At least one parent available per family; At least 3 siblings per family if only one parent available for studyAmong the 81 families, 18 families had both parents with documented NIDDM, 46 families one parent affected, and 17 families both parents clinically normal. A total of 432 individuals from these 81 families were selected in this study, including 152 parents and 280 non-diabetic offspring defined by both oral glucose tolerance tests (OGTT) and steady-state plasma glucose tests (SSPG).

Basic clinical data, such as age, gender, weight, height, waist-hip ratio, age of onset of NIDDM, and medical history were collected during the initial hospital visit for each individual. BMI was used as a general index of obesity as calculated by weight in kg divided by height (in meters) squared. In addition, the role of abdominal obesity was estimated by determining the ratio of abdominal to hip girth (WHR for waist-hip ratio). Waist circumference was measured at the level of the umbilicus and hip circumference determined over the widest part of the gluteal area.

Systolic and diastolic pressure were measured in the sitting position three times at 20-minute intervals by an experience nurse both by conventional sphygmomanometry and by an automatic portable device based on oscillometric technology. The mean value of these three data points was used to determine the level of systolic and diastolic pressure, respectively.

Glucose and insulin response to an oral load of glucose was determined by an OGTT. Each study subject was given a 75g oral glucose (Glucola) to drink, and blood samples were collected 10 minutes before glucose intake, at the time of glucose intake (0 min) and at 30, 60, 90, 120, and 180 min after the oral load of glucose. Plasma glucose and insulin levels were measured in these samples using an enzymatic colormetric method and automated immunoassays.

After an overnight fast, intravenous catheters are placed in each arm of the study subject. Blood samples were collected from one arm for measurements of plasma glucose and insulin concentration and the other arm was used for administration of test substances. Sandostatin was administered at 25ug/h in a solution containing 2.5% (w/v) human serum albumin by a Harvard infusion pump to suppress endogenous insulin secretion. Simultaneously, insulin and glucose were infused at 25mU/m2/min, respectively. Blood samples (7 ml each) were collected at -10 min, 0 min, before the initiation of the infusion, every half an hour until 150 min into the study, and then every 10-min until 180 min. Insulin concentrations typically reach plateau by 60 min, whereas glucose concentrations reach plateau after 120 min. The four values obtained from 150, 160, 170 and 180 min were averaged and considered to represent the steady-state plasma glucose (SSPG) and steady-state plasma insulin (SSPI) concentrations achieved during the infusion. Since SSPI concentrations were comparable in all individuals, both qualitatively and quantitatively, the glucose infusion rate identical, the magnitude of the resultant SSPG concentration provides a quantitative estimate of the effectiveness of insulin in disposal of a glucose load, i.e., the higher the SSPG, the more insulin resistant the person.

Blood samples (15 ml each) were collected on two different days after overnight fasting, once on the day of OGTT, and the second on the day of SSPG test. Lipid and lipoprotein measurements were performed using standard enzymatic methods.

Cell lines were established from B-lymphocyte cell lines from 245 study subjects using standard EB virus transformation.

Example 2:Identification of IRM SequencesBased on the phenotypic analysis described supra, six subjects were identified as having an extreme insulin resistance ("eIR") phenotype, and six subjects were identified as having an extreme insulin sensitivity ("eIS") phenotype. Subjects were assigned to the eIR group if they met the following criteria: OGTT Glu at 120m > 140 mg/dl; SSPG mean > 250 mg/dl; OGTT Ins at 60m > 100 µ IU/ml Et; OGTT Ins at 120 m > 100 IU/ml. Subjects were assigned to the eIS group if they met the following criteria: OGTT Glu at 120m < 100 mg/dl; SSPG mean < 120 mg/dl; OGTT Ins at 60m < 60 µ IU/ml OR OGTT Ins at 120 m < 40 µIU/ml.

EBV-transformed B-lymphocyte cell lines from each subject were cultured in RPMI-1640 media containing 10% fetal bovine serum (FBS) in a 37C, 5% CO₂ incubator for about two weeks. These cell lines were transferred to RPMI-1640 containing 3% of FBS for 72 hours, and switched to RPMI-1640 containing 3% of FBS and either 15 µ IU/ml of insulin or 100 µ IU/ml of insulin, and incubated for another 72 hours. At the time of RNA extraction, these cell lines from each IR and IS group were grown under the same culture conditions to the same passages. Total RNA from each cell lines was extracted using standard Trizol method (Gibco-BRL). Equal amounts of total RNA from the 6 eIR cell lines were pooled to form the IR-RNA pool and equal amounts of total RNA from 6 eIS cell lines were pooled to form the IS-RNA pool. Differently labeled probes were prepared by reverse transcription with oligo-dT primer to specifically amplify mRNA from the pooled total RNA. The IR pool was labeled with Cy5-deoxyuridine triphosphate (dUTP) and the IS-pool with Cy3-dUTP via reverse transcription.

The labeled cDNAs from each pool were mixed and simultaneously hybridized to microarrays containing approximately 10,000 expressed sequence tags from genes expressed in blood cells (see PCT publication WO 00/40749) or microarrays containing approximately 40,000 ESTs from genes expressed in variety of human tissues (http://genome-www4.stanford.edu/cgi-bin/sfgf/home.pl/). cDNA labeling, microarray hybridization, and washing were performed according to standard protocols for CMT-GAPS slides provide by manufacture Corning (http://www.corning.com/CMT/TechInfo/PDFs/ cmt_amino_silane_im.pdf). Differentially expressed genes were identified by scanning the microarrays using a GenePix 4000A scanner with GenePix Pro 3.0 microarray analysis software from Axon Instruments, Inc, Foster City, Calif.. The scan image allows identification of genes whose mRNA are more abundant in IR pool as red spots (Cy5) and genes whose mRNA are more abundant in the IS pool as green spot (Cy3). Yellow spots suggest no significant variation in gene expression between IR- and IS-pools for those specific cDNA spots.

Example 3:Additional Analysis of IRM ExpressionA number of assays are used for further analysis of the IRM genes of the invention. These include: (a) Northern analysis experiments in which expression of an IRM gene in EBV-transformed B lymphocyte cell lines derived from eIS and eIR populations is determined. The Northern analysis can use RNA pooled from multiple cell lines or obtained from an individial cell line.

(b) Northern analysis experiments in which expression of an IRM gene in individuals of known insulin resistance status (e.g., having an eIS or eIR phenotype) is determined. The Northern analysis can use RNA pooled from several individuals or obtained from a single individial.

(c) Quantative real time PCR (qRT-PCR) in which expression of an IRM gene in EBV-transformed B lymphocyte cell lines derived from eIS and eIR populations is determined. The qRT-PCR can be applied to RNA pooled from multiple cell lines or obtained from an individial cell line.

(e) Quantative real time PCR in which expression of an IRM gene in individuals of known insulin resistance status (e.g., having an eIS or eIR phenotype) is determined. The qRT-PCR can use RNA pooled from several individuals or obtained from a single individial.

The practice of each of these assays will be well within the capability of one of ordinary skill following the guidance of this specification, and at least some of the additional assays have been carried out for many of the IRM genes disclosed herein. Each of the assays is described in general terms below:"Flip-Dye"array hybridization. Differential expression of sets of IRM genes can be confirmed or detected using additional rounds of hybridization of probes from eIR and eIS cell lines to array cDNA sequences, including rounds of hybridization using the "flip-dye"technique in which the labels used for each probe preparation are reversed. See Wang et al., 2000, Nat Biotech.18:457-59. For example, the eIR cDNA pool labeled with Cy5 (red) in a first experiment can be labeled with Cy3 (green), and, in a second experiment, eIS cDNA pool originally labeled with Cy3 can be labeled with Cy5. Using this method, if a gene "X"(that hybridizes to immobilized probe "X"") is over-expressed in the eIR cell lines, the location of X" should appear as a red spot on the array in the first experiment and as a green spot on the array in the second experiment.

Northern Analysis. Northern analysis to monitor differential expression in populations can be carried out using probes that hybridize to IRM genes. Methods of carrying out Northern analysis are well known (see, e.g., Sambrook, supra). In one assay, total RNA is prepared from EBV-transformed B-lymphocyte cell lines from subjects with an eIS or eIR phenotype. Alternatively, RNA is prepared from blood samples of subjects with an eIS or eIR phenotype. In either case, the samples or RNA from the samples can be analysed individually (provided a sufficient quantity of RNA can be obtained) or pooled.

20 ug of total RNA for each sample is loaded into the wells of a 1% denaturing agarose gel (2.2M formaldehyde, 20mM MOPS(3-[N-morpholino]propanesulfonic acid), 2mM sodium acetate, 1mM EDTA, and 5ng/ml ethidium bromide). Electrophoresis is performed at 100volts for 4 hours. After running the gel, a photograph of the gel is taken under UV to examine the integrity and the consistency of loading quantity of RNA samples. The RNA in the gel is transferred to nylon filter (Hybond-N, Amersham) overnight and fixed by baking at 80^{o}C for 2 hours. Transferred RNA was prehybridized and then hybridized with labeled probes.

³²P-labeled IRM probes are prepared using a random priming kit (High Prime, Roche Inc.) and fragments of the IRM genes. For example, a purified 500bp DNA fragment derived from PCR amplification of IMAGE clone 1909455 is used as a probe to detect RNA level of immunoglobulin kappa chain precursor V-III gene. Pre-hybridization is performed in Church buffer (0.5M sodium phosphate buffer, 7% SDS, 10mM EDTA) at 65^{o}C for 4 hours in a rotisserie hybridization oven. Hybridization with the probe labeled with ³²P-dCTP is performed under the same condition for 16 hours. After washing twice with 2 × SSC (300 mM sodium chloride, 30 mM trisodium citrate, pH 7.0), 0.1% SDS for 15 min at room temperature and once with 0.1 ×SSC, 0.1% SDS for 30 min at 50^{o}C, the filters are dried and autographed at 70^{o}C using BioMaxMR films (Kodak) for 3 days.

Each Northern blot film is scanned and analyzed using a gel documentation and analysis system, Alpha Imager 2200 (Alpha Innotech Corp), according to the manufacture"s instructions. Signal intensity for each band, as measured by intensity unit relative to background (RIU) is determined, and the mean intensity of the eIS samples is used as a reference value. The fold difference, as measured by eIR intensity divided by mean eIS intensity is determined for each of the IR samples. A difference of 2-3-fold is usually considered significant, depending on the standard deviation among the eIS samples.

Quantative real time PCRA variety of "real time quantitative PCR"methods can also be utilized to determine the quantity of IRM mRNA present in a sample. See, e.g., Higuchi et al., 1992, Biotechnology 10:413-17; Weis et al., 1992, Trends in Genetics 8:263-64; Ausubel et al., supra, Current Protocols in Molecular Biology; Sambrook, et al., supra; Bulletin #2 for ABI PRISM 7700 Sequence Detection System (ABI). In one embodiment, equal amounts of total RNA isolated from 6 unrelated eIR or eIS individuals is pooled for analysis. Five micrograms of the pooled total RNA is used for cDNA synthesis. The first strand synthesis of cDNA is made by SuperScript reverse transcriptase (Invitrogen) with random hexamers. After the inactivation of reverse transcriptase by heat denaturation, the sample is digested by RnaseH to eliminate RNA. The cDNA is then purified away from primers, unreacted dNTPs and enzymes using the Qiaquick DNA purification kit (Qiagen). The final yield of the reverse transcription reaction is determined by OD measurement and 260nm, and the cDNA was diluted into 1 ng/μl.

In order to measure the expression level of the target genes, SYBR-green real-time quantitative PCR assays is utilized. The PCR reaction consists of 300nM of the primer pairs, 10ng of the cDNA, and 2x SYBR green PCR ready mix (Applied Biosystems, Foster City, CA) in a final volume of 50μl. The PCR reaction and real time detection was performed on the ABI"s Prism Sequencing Detection System 7700 (Applied Biosystems, Foster City, CA). The PCR cycle was set for follows: 50^{o}C for 2 minutes, 95^{o}C for 10 minutes, followed by 40 cycles of 95^{o}C, 15 second, and 58^{o}C for 60 seconds. The signal was collect during the real time run and at the endpoint. The sequence was analyzed by ABI"s sequencing detection software 1.6.

The expression level of a gene target is translated to Ct (cycle threshold). Higher expression level is translated into an earlier Ct (smaller number), and a lower level translated into a later Ct (large number). The same gene expressed in two different test samples (e.g. eIR cell lines and eIS cell lines, blood from an eIR individual and blood from an eIS individual, etc.) has two CT"s. The difference of the two CT"s (delta Ct,) is used to calculate the differential expression of the gene in two different samples. In the testing range (15 - 35Ct), one Ct represents a two-fold difference.

Example 4: Quantitative and Diagnostic IRM Assays This example describes exemplary results of additional analysis of insulin resistance markers. Additional hybridization assays were carried out using as described in Example 2, using the flip dye method. Differential expression of IRM 120 was detected in 6 of the 8 rounds of hybridization.

Assays were carried out using qRT-PCR to determine IRM expression in blood. Equal amounts of total RNA isolated from fasting blood samples collected from 9 unrelated eIR or eIS individuals were pooled for analysis. Five micrograms of the pooled total RNA from each group was used for cDNA synthesis. The first strand synthesis of cDNA was made by SuperScript reverse transcriptase (Invitrogen) with random hexamers. After the inactivation of reverse transcriptase by heat denaturation, the sample was digested by RnaseH to eliminate RNA. The cDNA was then purified from primers, unincoporated dNTPs and enzymes using the Qiaquick DNA purification kit (Qiagen).

To measure the expression level of the IRM 120, SYBR-green real-time quantitative PCR assays were used. The PCR reaction consisted of: 300nM of the primer pairs, 10ng of the cDNA, and 2x SYBR green PCR ready mix (Applied Biosystems, Foster City, CA) were in a final volume of 50μl. The PCR reaction and real time detection was performed on the ABI"s Prism Sequencing Detection System 7700 (Applied Biosystems, Foster City, CA). The PCR cycle was set for follows: 50^{o}C for 2 minutes, 95^{o}C for 10 minutes, followed by 40 cycles of 95^{o}C, 15 second, and 58^{o}C for 60 seconds. The signal was collect during the real time run and at the endpoint. The sequence was analyzed by ABI"s sequencing detection software 1.6.

The expression level of a gene target was translated to Ct (cycle threshold). A higher expression level is translated into an earlier Ct (smaller number), and a lower level translated into a later Ct (large number). The same gene expressed in two different test samples (e.g. eIR and eIS) has two CT"s. The difference of the two CT"s (delta Ct,) is used to calculate the differential expression of the gene in two different samples. In the testing range (15 - 35Ct), one Ct represents a two-fold difference (ABI user bulletin #2).

Additional hybridization assays were carried out using quantitative RT-PCR using blood from eIR and eIS phenotype individuals. RNA extraction from 10 ml fasting blood of eIR and eIS individuals was performed using the TRIZOL RNA isolation protocol (GIBCOBRL, Cat# 15596-018), and purified total RNA re-suspended in 100 ul of DEPC-treated Tris buffer (10mM, pH 7.0). cDNA synthesis was performed individually using approximately 0.5 ug of blood RNA from 6 unrelated eIR individuals. For comparison, cDNA synthesis was also performed using equal amount of total RNA pooled from 9 eIS individuals (eIS-pool). The first strand synthesis of cDNA was made by SuperScript reverse transcriptase (Invitrogen) with random hexamers. After the inactivation of reverse transcriptase by heat denaturation, the sample was digested by RnaseH to eliminate RNA. The cDNA was then purified away from primers, unreacted dNTPs and enzymes using the Qiaquick DNA purification kit (Qiagen). In order to measure the expression level of the IRM120 genes, SYBR-green real-time quantitative PCR assays was performed using primers specific for IRM120 ( Forward primer : 5"- CAG AAG GAA ATT AAG CAA ACA-3"; Reverse primer: 5"-CCG TAT ATG GCA ATT CAA TAA-3"; Size of amplicon = 98 bp). The PCR reaction consisted of: 300nM of the primer pairs, 10ng of the cDNA, and 2x SYBR green PCR ready mix (Applied Biosystems, Foster City, CA) were in a final volume of 50μl. The PCR reaction and real time detection was performed on the ABI"s Prism Sequencing Detection System 7700 (Applied Biosystems, Foster City, CA). The PCR condition was set for follows: 50^{o}C for 2 minutes, 95^{o}C for 10 minutes, followed by 40 cycles of 95^{o}C, 15 second, and 58^{o}C for 60 seconds. Quantitative RT-PCR was performed in triplicate for each sample, and data was collect during the real time run and at the endpoint. The sequence was analyzed by ABI"s sequencing detection software 1.6. In addition, at the end of each run, the size and quality of each amplicon was verified using side-by-side gel electrophoresis (3% agarose gel in electrophoresis tank containing 1X TBE, run at 100 volts for 45 minutes) with 1 ug DNA size standard (Cat# E-3048-1, ISC BioExpress) to ensure the absence of non-specific amplification and/or primer-dimer band.

The expression level of the gene target was translated to Ct (cycle threshold), which is a user defined threshold at which the fluorescence intensity due to double stranded DNA binding to SYBR green is 10x the background value (determined earlier in the PCR reaction). A higher expression level is translated into an earlier Ct (smaller number), and a lower expression level translated into a later Ct (larger number). The analyte Ct is initially normalized vs. a control gene, in this case GAPDH. The difference between the analyte and control Ct is defined as the delta Ct. This value is then compared with a predetermined standard, in this case the eIS pool, to obtain the Ct value. In the testing range (15 - 35Ct), one Ct represents a two-fold difference. Given these assumptions, one can use 2⁻^{Ct} to obtain the relative expression of an analyte (ABI user bulletin #2).

These data indicate that IRM120 is uniformly underexpressed in eIR patient blood compared to eIS patient blood. This demonstrates that IRM120 is useful as a diagnostic marker and in drug screening applications.

Notably, both IRM50 and IRM120 are contained in a genomic segment approximately 8000 bp in a 182,943 bp BAC with GenBank Acc # of AC016251.9 (corresponding roughly to bases 3000-11000 of the BAC). There are four known exons of IRM120, mapped to nt996910386 (exon 1), nt 9603-9911 (exon2), nt7547-8076 (exon 3), and nt4375-4804 (exon4) of the BAC sequence. IRM5 EST sequence is mapped to nt3682-4016, approximately 350 bp down stream of exon 4 of IRM120. In addition to physical linkage of IRM 120 and 50, both are down-regulated to similar extent in cell lines established from eIR individuals as compared to that from eIS subjects. The data suggest that IRM50 exhibits the same uniformly underexpression in eIR patient blood compared to eIS patient blood as described supra for IRM120. Further, these data suggest that IRM50 may be a splice variant of IRM120. In addition, cDNA clone AK025842 (1590 bp; hereinafter designated IRM 393) is mapped between exon 3 and 4 of IRM120. Furthermore, several IMAGE clones (e.g., Acc#4849984, 4862951, 731736, 4900978,5199490, 5200043) are also mapped to the 8000bp genomic segment of the BAC AC016251.9 containing IRM50 and IRM120. Thus, the BAC sequence, clone AK025842, or the IMAGE clones also are insulin resistance markers. In various embodiments, probes that hybridize to the BAC sequence, clone AK025842, or the IMAGE clones, and polynucleotides and proteins encoded by the genes corresponding to these sequences, may be used in the diagnostic, prognostic, screening, and other methods disclosed herein.

***It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and accession numbers (including both polynucleotide and polypeptide sequences and corresponding annotations as of the filing and/or priority application filing dates) cited herein are hereby incorporated by reference in their entirety for all purposes to the same extent as if each individual publication, patent or patent application were specifically and individually indicated to be so incorporated by reference.

Claims

1.A method for diagnosing for insulin resistance (IR), an IR-related condition, or susceptibility to IR or an IR-related condition in a subject, said method comprising detecting a difference in expression of at least one insulin resistance marker (IRM) listed in Table 1 in a biological sample from the subject, compared to the level of expression of the IRM characteristic of expression in a similar biological sample in a reference population of individuals who are not insulin resistant.

2.The method of claim 1 wherein the population of individuals who are not insulin resistant have an extreme insulin sensitivity (eIS) phenotype.

3.The method of claim 1 wherein an increase in expression of the IRM is diagnostic of insulin resistance (IR), an IR-related condition, or susceptibility to IR or an IR-related condition in the subject
4.The method of claim 1 wherein an decrease in expression of the IRM is diagnostic of insulin resistance (IR), IR-related conditions, or susceptibility to IR or IR-related conditions in the subject.

4. 5.The method of claim 1 wherein the biological sample is blood or a blood fraction.

5. 6.The method of claim 5 wherein the biological sample comprises B-lymphocytes.

6. 7.The method of claim 1 wherein the level of expression of the IRM is determined by detecting an IRM RNA.

7. 8.The method of claim 7 wherein detecting the RNA comprises hybridizing a probe derived from RNA of the subject to an immobilized polynucleotide that hybridizes to an IRM gene listed in Table 1, and detecting the formation of a hybridization complex.

8. 9.The method of claim 8 wherein that comprises hybridizing RNA of the subject, or a probe derived from RNA of the subject, to an array of immobilized polynucleotides, wherein said immobilized polynucleotides comprise polynucleotides that hybridize to at least two different IRM genes listed in Table 1.

9. 10.The method of claim 7 wherein detecting the RNA comprises hybridizing a cDNA probe to a plurality of immobilized polynucleotide.

10. 11.The method of claim 7 wherein the RNA encoded by the IRM is isolated from a blood sample from the subject.

11. 12.The method of claim 1 wherein the level of expression of said at least one IRM is determined by detecting a polypeptide encoded by an IRM gene listed in Table 1.

12. 13.The method of claim 1 wherein the step of detecting a difference in expression compared to the level of expression of the IRM characteristic of expression in a similar biological sample in a reference population of individuals who are not insulin resistant comprises determining that the level of expression is similar to the level of expression of the IRM characteristic of expression in a similar biological sample in a reference population of individuals who are insulin resistant.

13. 14.The method of claim 13 wherein the population of individuals who are insulin resistant have an extreme insulin resistance (eIR) phenotype.

14. 15.The method claim 1 further comprising identifying the subject as a patient at risk for insulin resistance based the medical history of the subject or the subject"s family prior.

15. 16. The method of claim 1 wherein a difference in expression of IRM 120 or IRM 50 is detected.

16. 17.A method of diagnosing an individual as insulin resistant or at increased risk for developing insulin resistance comprising:
(a) obtaining a biological sample taken from the subject, and
(b) comparing the expression level of a panel of at least 3 insulin resistance markers listed in Table 1 in the sample to a reference value representative of expression in a population of individuals of a known insulin resistance status,
wherein the individual is diagnosed as insulin resistant or at risk for developing insulin resistance when
(i) the expression level of at least 50% of the at least 3 insulin resistance markers is not statistically different to reference value, if the reference value is characteristic of expression in a population of subjects who are insulin resistant or
(ii) the expression level of at least 50% of the at least 3 insulin resistance markers at least 3 IRM genes is statistically different from a reference value, if the reference value is characteristic of expression in a population of subjects who are not insulin resistant.

17. 18.The method of claim 17 wherein population of subjects who are insulin resistant have an eIR phenotype.

18. 19.The method of claim 17 wherein population of subjects who are not insulin resistant have an eIS phenotype.

19. 20.A device for assaying for expression of a gene associated with insulin resistance comprising at least one polynucleotide probe that hybridizes to an IRM listed in Table 1 is immobilized, wherein the substrate comprises fewer than 4000 distinct polynucleotide probes.

20. 21.The devise of claim 20 wherein said substrate comprises fewer than 100 distinct polynucleotide probes.

21. 22.The device of claim 21 wherein the substrate comprises fewer than 10 distinct polynucleotide probes.

22. 23.The device of claim 21 that comprises probes that hybridize to at least four different IRM genes.

23. 24.The device of claim 21 wherein at least 10% of the immobilized probes are polynucleotides that hybridize to a IRM gene product.

24. 25.The device of claim 21 wherein the polynucleotides are immobilized on a glass slide.

25. 26.The device of claim 21 comprising at least one polynucleotide probe that hybridizes to IRM 120 or IRM 50.

26. 27.The method of claim 8 wherein the immobilized polynucleotide is immobilized on a device of claim 21.

27. 28.A method of screening for an agent to determine its usefulness in treating insulin resistance comprising
a) providing a cell expressing at least one insulin resistance marker (IRM) listed in Table 1;
b) contacting the cell with a test agent; and
c) determining whether the level of expression of an IRM is changed in the presence of the test agent, wherein a change is an indication that the test agent is useful in treatment of insulin resistance.

28. 29.The method of claim 28 wherein the cell is a cultured cell.

29. 30.The method of claim 29 wherein the cell is a primary culture or an established cell line.

30. 31.The method of claim 30 wherein the cell is selected from the group consisting of 3T3-L1 adipocytes; CHO cells; L6 rat skeletal myotubes; mouse macrophage RAW cells; Jurkat cells; PC12 (rat neuronal) cells; Hela cells; HEP G2 cells; Burkitt's lymphoma cell line Raji; Burkitt's lymphoma cell line Daudi; B-PLL cells line (p11A-1-1); B-ALL cell line MOLT-3 and B-ALL cell line MOLT-4.

31. 32. The method of claim 30 wherein the cell is selected from the group consisting of cell lines or primary cultures from patients with Burkitt's lymphoma, B-cell prolymphocytic leukemia, B-cell chronic lymphoblastic leukemia, or B-cell acute lymphoblastic leukemia.

32. 33.The method of claim 29 wherein the cell is an EBV-transfomed B-lymphocyte.

33. 34.The method of claim 33 wherein a change in the level of expression of an RNA is determined.

34. 35.The method of claim 28 wherein a change in the level of expression of a protein encoded by an IRM gene is determined.

35. 36.The method of claim 29 comprising determining for at least 2 insulin resistance markers whether or not the level of expression is changed in the presence of the test agent, wherein a change in the level of expression of at least one IRM is an indication that the test agent is useful in treatment of insulin resistance.

36. 37.The method of claim 36 comprising determining the level of expression of at least 5 insulin resistance markers.

37. 38.The method of claim 34 wherein the level of expression is determined using an amplification assay.

38. 39.The method of claim 34 wherein the level of expression is determined using a hybridization assay.

39. 40.The method of claim 28 further comprising administering the agent to an animal to determine whether the animal"s response to insulin is affected by the agent.

40. 41.The method of claim 40 wherein the animal is a rodent.

41. 42.The method of claim 28 wherein the cell expresses at least one of IRM 120 and IRM 50.

42. 43.A method of screening for an agent to determine its usefulness in treating insulin resistance comprising
a) providing a composition comprising an IRM protein,
b) contacting the composition with a test agent
c) determining whether the activity of the IRM protein is changed in the presence of the test product
wherein a change is an indication that the test agent is useful in treating insulin resistance.

43. 44.A method of screening for an agent to determine its usefulness in treating insulin resistance comprising (a) contacting a polypeptide encoded by an IRM gene, or a cell expressing said polypeptide with a test compound, wherein said polypeptide has a detectable biological activity; and (b) determining whether the level of biological activity of the protein is changed in the presence of the test agent, wherein a change is an indication that the test agent is useful in treatment of insulin resistance.

44. 45.A method of screening for an agent to determine its usefulness in treating insulin resistance comprising
a) contacting a polypeptide encoded by an IRM gene, or a cell expressing said polypeptide with a test compound; and
b) determining whether the polypeptide binds to the test compound, wherein binding is an indication that the test agent is useful in treatment of insulin resistance.

45. 46.A method of preparing a medicament for use in treating insulin resistance or an IR related condition comprising
a) determining that an agent is useful for treatment of insulin resistance using the method of claim 28, and
b)formulating the agent for administration to a primate.

46. 47.A method of screening for an agent for use in treating insulin resistance comprising
a) determining that an agent is useful for treatment of insulin resistance using the method of claim 28, and
b)adminnistering the agent to a nonhuman animal to determine the effect of the agent.

47. 48.A method of treating insulin resistance in a mammal, comprising administering an effective amount of an agent that modulates expression of an insulin resistance marker listed in Table 1.

48. 49.The method of claim 48 wherein the agent modulates expression of IRM 120 or IRM 50.

49. 50.A method for identifying a polymorphism associated with an insulin resistance (IR) phenotype or risk of developing insulin resistance comprising comparing the sequence of an IRM gene listed in Table 1 in a biological sample from an insulin resistant subject with sequence of the IRM gene in a biological sample from a non-insulin resistant subject.

50. 51.The method of claim 50 wherein the non-insulin resistant subject has an eIS phenotype.

51. 52.The method of claim 50 wherein the insulin resistant subject has an eIR phenotype.

52. 53.A method of determining whether an individual is at risk of developing insulin resistance or whether said individual suffers from insulin resistance comprising the steps of:
(a) obtaining a nucleic acid sample from said individual; and
(b) determining whether the nucleotides present at one or more IRM genes are indicative of a risk of developing insulin resistance.

53. 54. A method of detecting an association between a genotype and an insulin resistance phenotype, comprising the steps of:
(a) genotyping at least one IRM gene in a first population having a first insulin resistance phenotype;
(b) genotyping said IRM gene in a second population having a second insulin resistance phenotype different from the first insluin resistance phenotype; and
(c) determining whether a statistically significant association exists between said genotype and said phenotype.

54. 55.The method of claim 54 wherein the first population is eIS and the second population is eIR.

55. 56.A method of estimating the frequency of a haplotype for a set of nucleotide polymorphisms markers a population, comprising:
(a) identifying at least a first nucleotide polymorphism in an IRM gene listed in Table 1 for individuals in a population;
(b) identifying a second nucleotide polymorphism in an IRM gene for individuals in a population, wherein the second IRM gene is the same or different from the first IRM gene; and
(c) applying an haplotype determination method to the identities of the nucleotide polymorphisms determined in steps (a) and (b) to obtain an estimate of said frequency.

56. 57.A method of detecting an association between a haplotype and a phenotype, comprising the steps of:
(a) estimating the frequency of at least one haplotype in first population having a first insulin resistance phenotype according to the method of claim 56;
(b) estimating the frequency of said haplotype in a a second insulin resistance phenotype different from the first insulin resistance phenotype according to the method of claim 56; and
(c) determining whether a statistically significant association exists between said haplotype and the first insulin resistance phenotype.

57. 58.A method of claim 57 wherein the first insulin resistance phenotype is eIR
59.A method of claim 57 wherein the insulin resistance phenotype is eIS.

58. 60.A method for identifying genes associated with a disease state comprising
(a) identifying a first population of human subjects, wherein said subjects suffer from, or are at high risk of, developing the disease;
(b) identifying a second population of human subjects, wherein said subjects do not have and are at low risk of developing the disease; and
(c) obtaining cell lines derived from B lymphocytes from each of the subjects in the first and second populations
(ii) comparing the expression of RNAs in the cell lines of the first population and the cell lines in the second population, thereby identifying RNAs differentially expressed in the first population compared to the second population
wherein said RNAs differentially expressed in the first population compared to the second population are encoded by genes associated with a disease state.

59. 61.The method of claim 60 wherein the cell lines are established by transformation with Epstein Barr virus.

60. 62.The method of claim 60 wherein the first and second populations each comprise at least 3 individuals.

61. 63.The method of claim 60 wherein the first population is an extreme insulin resistant population and the second population is an extreme insulin sensitive population, or the first population is an extreme high HDL population and the second population is an extreme low HDL population, or first population is an extreme obese/high body mass population and the second population is an extreme lean/low body mass population.
